(19) United States
(12) Patent Application Publication
(10) Pub. No.: US 2024/0299595 A1
(43) Pub. Date: Sep. 12, 2024

(54) CANCER THERANOSTIC COMPOSITIONS COMPRISING BIGUANIDE COMPLEXES OF GROUP 7 TRANSITION METALS AND USES THEREOF

(71) Applicant: BOARD OF REGENTS, THE UNIVERSITY OF TEXAS SYSTEM, Austin, TX (US)

(72) Inventors: Chun LI, Missouri City, TX (US); Chiyi XIONG, Cypress, TX (US)

(73) Assignee: BOARD OF REGENTS, THE UNIVERSITY OF TEXAS SYSTEM, Austin, TX (US)

(21) Appl. No.: 18/549,663

(22) PCT Filed: Mar. 9, 2022
(86) PCT No.: PCT/US2022/019517

§ 371 Date: Sep. 8, 2023

Related U.S. Application Data
(60) Provisional application No. 63/158,750, filed on Mar. 9, 2021.

(51) Int. Cl. A61K 51/04; A61K 45/06; A61P 35/00; C07F 13/00

(52) U.S. Cl. CPC A61K 51/0406; A61K 45/06; A61P 35/00; C07F 13/00; A61K 2121/00; A61K 2123/00

(57) ABSTRACT

Described herein is a composition including a compound according to Formula I: wherein M is a Group 7 transition metal. R1 is selected from the group consisting of methyl, ethyl, propyl, butyl, and phenethyl; R2 is selected from the group consisting of —H and methyl; R3 is selected from the group consisting of —H, methyl, ethyl, propyl, butyl, and phenethyl; and R4 is selected from the group consisting of —H, methyl, and -L-Z, wherein L is a linker and Z is a targeting unit. Also disclosed are methods including administering, to a patient suffering from a cancer or suspected of suffering from a cancer, the composition. Further disclosed are kits comprising the composition and instructions to perform the method.

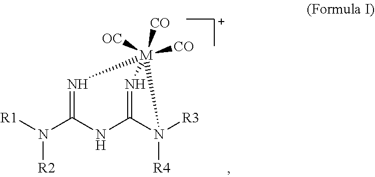

### CROSS-REFERENCE TO PRIORITY APPLICATION

This application claims priority to U.S. Provisional Application No. 63/158,750, filed Mar. 9, 2021, which is incorporated herein by reference in its entirety.

### FIELD

The present disclosure relates generally to the field of cancer diagnosis and therapy. More particularly, it concerns compositions comprising biguanide complexes of Group 7 transition metals and uses thereof in cancer diagnosis and cancer therapy.

### BACKGROUND

Metal-based compounds have been used successfully in cancer therapy, as demonstrated by the introduction of platinum-based drugs (i.e., cisplatin, carboplatin) to clinics. These agents act by damaging DNA in tumor cells; however, they are significantly toxic to healthy cells. Another obstacle to successful clinical translation of anticancer drugs in general, and metal-based agents in particular, is the difficulty in obtaining real time data on the tumor deposition and biodistribution of these agents to guide selection of dose and schedule for individual patients. Much interest has arisen recently for theranostic pairs, that is, structurally and/or chemically similarly compounds, one of which may provide diagnostic information regarding tumor deposition and biodistribution, and the other of which may provide therapeutic activity against the tumor.

The field of cancer theranostics is still in its early stages. Also, questions of toxicity to healthy tissues continually arise when metal-based diagnostic and/or therapeutic compounds are contemplated. Thus, there is a desire for metal-based diagnostic and/or therapeutic compounds with theranostic power and relatively low toxicity to healthy tissues.

### SUMMARY

The following presents a simplified summary of the invention in order to provide a basic understanding of some aspects of the invention. This summary is not an exhaustive overview of the invention. It is not intended to identify key or critical elements of the invention or to delineate the scope of the invention. Its sole purpose is to present some concepts in a simplified form as a prelude to the more detailed description that is discussed later.

Described herein is a composition, comprising a compound of Formula I:

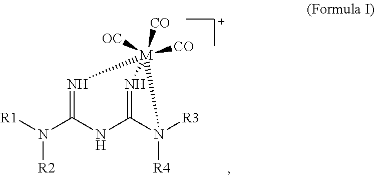

wherein M is a Group 7 transition metal, R1 is selected from the group consisting of methyl, ethyl, propyl, butyl, and phenethyl; R2 is selected from the group consisting of —H and methyl; R3 is selected from the group consisting of —H, methyl, ethyl, propyl, butyl, and phenethyl; and R4 is selected from the group consisting of —H, methyl, and -L-Z, wherein L is a linker (e.g., a carbonyl or carboxyl linker, an oligoethylene glycol, a biodegradable peptide, or the like) and Z is a targeting unit (e.g., a carbohydrate, such as glucose or a glucose derivative, a peptide, a peptoid, or the like). Optionally, M is selected from the group consisting of Tc and Re. In some examples, R1 is methyl, R2 is methyl, R3 is —H, and R4 is —H. In some examples, R1 is butyl, R2 is —H, R3 is —H, and R4 is —H. In some examples, R1 is phenethyl, R2 is —H, R3 is —H, and R4 is —H. In some examples, R1 is phenethyl, R2 is —H, R3 is —H, and R4 is -L-Z, wherein L is a carboxyl group and Z is a glucose containing moiety (e.g., 1,3,4,6-Tetra-O-acetyl-alpha-D-glucopyranose). Optionally, M is Re, R1 is phenethyl, and R2 is —H. The compositions described herein can further include a pharmaceutically-acceptable carrier (e.g., polyethoxylated castor oil, ethanol, aqueous saline solutions, or mixtures thereof).

Also described herein is a method of treating cancer in a subject, comprising administering to a patient suffering from a cancer or suspected of suffering from a cancer a composition comprising a diagnostically-effective amount or a therapeutically-effective amount of a compound of Formula I, wherein M is a Group 7 transition metal, R1 is selected from the group consisting of methyl, ethyl, propyl, butyl, and phenethyl; R2 is selected from the group consisting of —H and methyl; R3 is selected from the group consisting of —H, methyl, ethyl, propyl, butyl, and phenethyl; and R4 is selected from the group consisting of —H, methyl, and -L-Z, wherein L is a linker (e.g., a carbonyl or carboxyl linker, an oligoethylene glycol, a biodegradable peptide, or the like) and Z is a targeting unit (e.g., a carbohydrate, such as glucose or a glucose derivative, a peptide, a peptoid, or the like). Optionally, for example, when M is ^{99m}Tc, R1 is phenethyl, and R2 is —H, and the method further comprises imaging at least a portion of the patient's body after the administering. The imaging can optionally comprise computed tomography (CT) scanning. The method described herein can further comprise administering to the patient a cancer treatment modality other than the compound of Formula I. In some examples, the cancer treatment modality other than the compound of Formula I is selected from the group consisting of surgical resection, chemotherapy with a compound other than the compound of Formula I, immunotherapy, checkpoint inhibitor therapy, oncolytic virus therapy, thermal therapy, radiotherapy, and two or more thereof. In some cases, the patient is a human being.

Further described herein is a kit, comprising a composition, comprising a diagnostically-effective amount or a therapeutically-effective amount of a compound of Formula I, wherein M is a Group 7 transition metal, R1 is selected from the group consisting of methyl, ethyl, propyl, butyl, and phenethyl; R2 is selected from the group consisting of —H and methyl; R3 is selected from the group consisting of —H, methyl, ethyl, propyl, butyl, and phenethyl; and R4 is selected from the group consisting of —H, methyl, and -L-Z, wherein L is a linker (e.g., a carbonyl or carboxyl linker, an oligoethylene glycol, a biodegradable peptide, or the like) and Z is a targeting unit (e.g., a carbohydrate, such as glucose or a glucose derivative, a peptide, a peptoid, or the like); and instructions for use of the composition in a method comprising administering, to a patient suffering from a cancer or suspected of suffering from a cancer, the composition. In some cases, such as when M is ^{99m}Tc, R1 is phenethyl, and R2 is —H, the instructions further comprise instructions to image at least a portion of the patient's body after the administering. Optionally, the instructions further comprise instructions to administer, to the patient, a cancer treatment modality selected from the group consisting of surgical resection, chemotherapy with a compound other than the compound of Formula I, immunotherapy, checkpoint inhibitor therapy, oncolytic virus therapy, thermal therapy, radiotherapy, and two or more thereof

The details of one or more examples are set forth in the drawings and the description below. Other features, objects, and advantages will be apparent from the description and drawings, and from the claims.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific examples presented herein.

FIG. 1 presents a flowchart of a method in accordance with examples herein.

FIG. 2A depicts a synthesis scheme for Re-Phen and ^{99m}Tc-Phen, in accordance with examples herein.

FIG. 2B shows the HMBC (heteronuclear multiple bond correlation) NMR spectrum of Re-Phen, in accordance with examples herein.

FIG. 2C depicts high resolution mass spectroscopy characterization of Re-Phen, in accordance with examples herein.

FIG. 2D shows an HPLC chromatogram of Re-Phen, in accordance with examples herein.

FIG. 2E shows HPLC chromatogram of ^{99m}Tc-Phen before purification, in accordance with examples herein.

FIG. 2F shows HPLC chromatogram of ^{99m}Tc-Phen after purification, in accordance with examples herein.

FIG. 3A presents representative μSPECT/CT images of mice bearing orthotopic MiaPaca-2 tumors at 1 hour and 4 hours after IV administration of ^{99m}Tc-Phen, in accordance with examples herein.

FIG. 3B shows HPLC chromatograms of urine samples collected at 1 and 4 hours after IV injection of ^{99m}Tc-Phen, in accordance with examples herein.

FIG. 3C shows representative μSPECT/CT images of a live animal acquired at 1 hour after radiotracer injection and post-mortem imaging of the same mouse after removal of tumor/pancreas (n=3). Middle panel: photograph of the mouse with tumor/pancreas removed and placed alongside the euthanized animal.

FIG. 4A presents representative autoradiographs of pancreas from a tumor-bearing mouse and a healthy mouse.

FIG. 4B compares an autoradiograph of mouse pancreas with tumor with corresponding histological features of an adjacent slide stained with H&E.

FIG. 5A compares biodistribution of ^{99m}Tc-Phen in healthy mice and tumor-bearing mice at 1 hour and 4 hours after radiotracer injection. % ID/g, percent of injected dose per gram of tissue.

FIG. 5B presents pancreas-to-tissue ratios of ^{99m}Tc-Phen between healthy mice and tumor-bearing mice. Data are expressed as mean±standard deviation (n=4). *p<0.05, **p<0.01, ***p<0.001 (Student's t test).

FIG. 6A outlines an in vivo experiment assessing antitumor activity of Re-Phen against orthotopic Kras* murine PDAC tumor. Male C57BL/6 mice were inoculated in the pancreas with iKrasG12D murine tumor cells. Treatments were initiated on day 7 after tumor cells inoculation. Each mouse received 10 daily intraperitoneal injections of phenformin or Re-Phen at a dose of 50 mg/kg/injection (100 μL/mouse). Control mice received 50/50 (v/v) Cremorphor EL/absolute ethanol, further diluted at 1:4 (v/v) ratio with saline (100 μL/mouse) before injection.

FIG. 6B presents tumor weights recorded at the end of the study of the antitumor activity of Re-Phen against orthotopic Kras* murine PDAC tumor.

FIG. 6C shows images of all excised tumors from the 3 treatment groups of the study of the antitumor activity of Re-Phen against orthotopic Kras* murine PDAC tumor.

FIG. 6D presents animal body weights of the three treatment groups of the study of the antitumor activity of Re-Phen against orthotopic Kras* murine PDAC tumor.

FIG. 6E shows representative microphotographs of immunohistochemically stained tumor slices for Ki67 showing reduced tumor proliferation after treatments with phenformin or Re-Phen. Magnification of the original slides: ×200.

FIG. 7A shows AMPK and AMPK phosphoprotein expression levels analyzed by western blotting. R-actin was used as a loading control. Cells were treated with phenformin or Re-Phenformin overnight at the indicated concentrations for 24 hours.

FIG. 7B shows flow cytometry dot plots of PDAC cells treated with 250 μM phenformin or Re-phenformin for 24 hours. Vehicle (DMSO) treated cells were used as a control.

FIG. 8A shows changes of mitochondrial membrane potentials of cells after treatments with phenformin or Re-Phen at indicated concentrations using a JC-10 microplate assay kit. The fluorescent intensities for J-aggregates (Ex/Em=540/590 nm) and monomeric forms (Ex/Em=490/525 nm) of JC-10 were measured with a microplate reader. Fluorescence intensity ratio measured at 525 nm/590 nm represents the change of mitochondrial membrane potential. Data are presented as mean±SD (n=4). *p<0.05; **p<0.01; ***p<0.001.

FIG. 8B shows representative cell mito-stress test results showing changes in oxygen consumption rate (OCR) after 4-hour treatments with phenformin or Re-Phen using a Seahorse XF24e analyzer.

FIG. 8C shows OCR rates calculated from Seahorse experiments described in Example 1. The glycolytic ratio was obtained using a YSI metabolic analyzer after treatments with phenformin (30 μM) or Re-Phen (30 μM) for 24 hours. Wells with culture media but without cells were used for baseline reading. Data are presented as mean±SD (n=3). *p<0.05; **p<0.01.

FIG. 8D shows increased ratios of NADH over total NAD (NADt=NAD+NADH) were observed in MiaPaca-2 and Kras* cells after treatments with Phen (100 μM) or Re-Phen (100 μM) for 3 hours or 12 hours. Data are presented as mean±SD (n=3). *p<0.05; ***p<0.001. Vehicle (DMSO) treated cells were as controls.

FIG. 9A shows levels of glutathione in vehicle, Phen (200 μM), or Re-Phen (200 μM)-treated MiaPaca-2 cells. Samples were measured by untargeted IC-MS after 12 hours of treatment (n=3).

FIG. 9B shows reactive oxygen species (ROS) measured by DCFDA assay. Decreased fluorescence intensity measured at Ex/Em 485/535 nm indicated decreased level of ROS. Data are expressed as mean±SD (n=4). ***p<0.001 compared to vehicle (DMSO) control.

FIG. 10A shows levels of metabolites in the glycolysis pathway in vehicle (DMSO), Phen (200 μM), or Re-Phen (200 μM)-treated MiaPaca-2 cells after 12 hours of treatment. Metabolites were extracted from lysed cells and samples were measured by untargeted IC-MS (n=3). Data are expressed as mean±SD (n=3). *p<0.05 compared to phenformin.

FIG. 10B shows levels of metabolites in the TCA cycle in vehicle (DMSO), Phen (200 μM), or Re-Phen (200 μM)-treated MiaPaca-2 cells after 12 hours of treatment. Metabolites were extracted from lysed cells and samples were measured by untargeted IC-MS (n=3). Data are expressed as mean±SD (n=3). *p<0.05 compared to phenformin.

FIG. 11A shows the impact of Phen and Re-Phen on the pyrimidine nucleotide pool.

FIG. 11B shows the impact of Phen and Re-Phen on the purine nucleotide pool.

FIG. 11C shows a scatterplot representing the pathway impact value and p-value from pyrimidine pathway topology analysis of the differentially expressed metabolites from Example 1. The size and shading of each node is based on its pathway impact value and p-value, respectively. Pathways with statistical significance (p<0.05) are shown in the circles with the densest shading.

FIG. 11D shows a scatterplot representing the pathway impact value and p-value from purine pathway topology analysis of the differentially expressed metabolites from Example 1. The size and shading of each node is based on its pathway impact value and p-value, respectively. Pathways with statistical significance (p<0.05) are shown in the circles with the densest shading.

FIG. 12 shows a general representation of Formula I, wherein M is a Group 7 transition metal.

FIG. 13A shows the pancreatic cell viability in percent as a function of concentration of Re-Phen after treatment for 72 hours.

FIG. 13B shows the pancreatic cell viability in percent as a function of concentration of phenformin after treatment for 72 hours.

FIG. 14A shows the RAW264.7 (mouse macrophages) viability in percent as a function of concentration when treated with Re-Phen or phenformin for 72 hours.

FIG. 14B shows the NIH3T3 (mouse embryonic fibroblasts) viability in percent as a function of concentration when treated with Re-Phen or phenformin for 72 hours.

FIG. 14C shows the hTERT-HPNE (immortalized human ductal endothelial cells) viability in percent as a function of concentration when treated with Re-Phen or phenformin for 72 hours.

FIG. 15 shows bar graphs comparing NADH-to-total NAD (NADt) ratio between Re-Phen and phenformin treated MiaPaca-2 and Kras cells after 3 hours and 12 hours.

FIG. 16 shows bar graphs comparing intracellular reactive oxygen species (ROS) after treatment with Re-Phen and phenformin in MiaPaca-2 and Kras cells.

FIG. 17A shows bar graphs comparing intracellular glutathione (GSH) levels after treatment with Re-Phen and phenformin in MiaPaca-2 and Kras cells.

FIG. 17B shows bar graphs comparing intracellular glutathione (GSH) to intracellular glutathione disulfide (GSSG) ratios after treatment with Re-Phen and phenformin in MiaPaca-2 and Kras cells.

FIG. 18 depicts a synthesis scheme for a Re-Phen-glucose conjugate.

FIG. 19A shows the ovarian cell viability in percent as a function of concentration of Re-Phen after treatment for 72 hours.

FIG. 19B shows the ovarian cell viability in percent as a function of concentration of phenformin after treatment for 72 hours.

While the subject matter disclosed herein is susceptible to various modifications and alternative forms, specific examples thereof have been shown by way of example in the drawings and are herein described in detail. It should be understood, however, that the description herein of specific examples is not intended to limit the invention to the particular forms disclosed, but on the contrary, the intention is to cover all modifications, equivalents, and alternatives falling within the spirit and scope of the invention as defined by the appended claims. Moreover, the stylized depictions illustrated in the drawings are not drawn to any absolute scale.

### DETAILED DESCRIPTION

Disclosed herein is a class of metal based compounds that can be used for the purpose of both diagnosis and therapy (theranostics). The proof-of-principle for metal based compounds have been demonstrated by successful introduction of platinum-based drugs to the clinics (i.e., cisplatin, carboplatin). However, these DNA-damaging agents have significant toxicity. One of the major obstacles to successful clinical translation of anticancer drugs in general and metal-based agents in particular is the difficulty in obtaining real time data on the tumor deposition and biodistribution of these agents to guide selection of dose and schedule for individual patients. The co-development of theranostic pairs represents a new paradigm in drug design and discovery. Disclosed herein are novel combinations of mitotropic agent (biguanides) and metal ions to achieve real time visualization of tumor uptake and biodistribution (with ^{99m}Tc-complexes) and with increased therapeutic effects (with Re-complexes). The technology can be further applied to therapeutic radionuclides of Re (e.g., ¹⁸⁸Re, a beta emitter) as well.

Various illustrative examples of the invention are described below. In the interest of clarity, not all features of an actual implementation are described in this specification. It will of course be appreciated that in the development of any such actual example, numerous implementation-specific decisions must be made to achieve the developers' specific goals, such as compliance with system-related, regulatory, and business-related constraints, which will vary from one implementation to another. Moreover, it will be appreciated that such a development effort might be complex and time-consuming but would nevertheless be a routine undertaking for those of ordinary skill in the art having the benefit of this disclosure.

The present subject matter will now be described with reference to the attached figures. Various structures, systems, and devices are schematically depicted in the drawings for purposes of explanation only and so as to not obscure the present disclosure with details that are well known to those skilled in the art. Nevertheless, the attached drawings are included to describe and explain illustrative examples of the present disclosure. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase.

As used herein, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only, or the alternatives are mutually exclusive. As used herein “another” may mean at least a second or more.

Throughout this application, any given numerical value includes the inherent variation of error for the device, or the method being employed to determine the value, or the variation that exists between study subjects or healthcare practitioners.

### I. Compounds

In one example, the present disclosure relates to a composition, comprising a compound of Formula I:

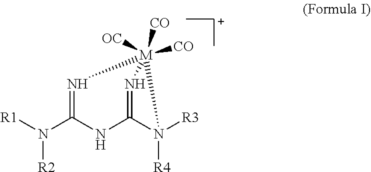

wherein M is a Group 7 transition metal, R1 is selected from the group consisting of methyl, ethyl, propyl, butyl, and phenethyl; R2 is selected from the group consisting of —H and methyl; R3 is selected from the group consisting of —H, methyl, ethyl, propyl, butyl, and phenethyl; and R4 is selected from the group consisting of —H, methyl, and -L-Z, wherein L is a linker (e.g., a carbonyl or carboxyl linker, an oligoethylene glycol, a biodegradable peptide, or the like) and Z is a targeting unit (e.g., a carbohydrate, such as glucose or a glucose derivative, a peptide, a peptoid, or the like). Generally, the linker can be or can include any structure that includes oligoethylene glycol, to modulate pharmacokinetics for example, or a peptide that can fragment upon encountering cathepsin enzymes in tumors. Optionally, the linker is a linker suitable for use in any antibody-drug conjugate. The targeting unit can be, for example, glucose, a peptide, or any other unit recognized by biomarkers and overexpressed in cancer cells or cancer stroma cells.

As is known, the Group 7 transition metals include manganese (Mn), technetium (Tc), rhenium (Re), and bohrium (Bh). M may be any isotope or mixture of isotopes of the Group 7 transition metal. In a particular example, M is selected from the group consisting of Tc and Re. In an even more particular example, M is selected from the group consisting of ^{99m}Tc or Re.

The selection of each of R1, R2, R3, and R4 may be made independently of the others. In one particular example, R1 is methyl, R2 is methyl, R3 is —H, and R4 is —H, i.e., the biguanide moiety of the compound is metformin (Structure I-A).

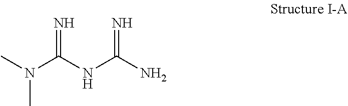

In another particular example, R1 is butyl, R2 is —H, R3 is —H, and R4 is —H, i.e., the biguanide moiety of the compound is buformin (Structure I-B).

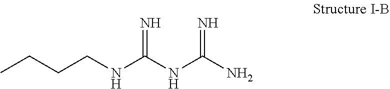

In yet another particular example, R1 is phenethyl, R2 is —H, R3 is —H, and R4 is —H, i.e., the biguanide moiety of the compound is phenformin (which may be termed “Phen”) (Structure I-C).

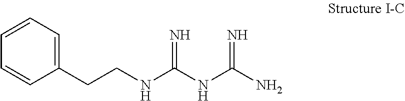 [SMILES: N=C(N)NC(=N)NCCc1ccccc1]

In still another example, R1 is methyl, R2 is methyl, R3 is methyl, and R4 is methyl, i.e., the biguanide moiety of the compound is metformin dimer (Structure I-D).

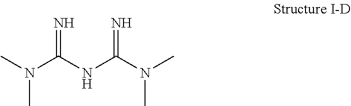

In another particular example, R1 is butyl, R2 is —H, R3 is butyl, and R4 is —H, i.e., the biguanide moiety of the compound is buformin dimer (Structure I-E).

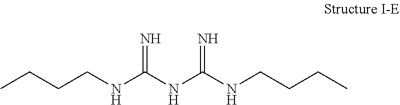

In yet an additional particular example, R1 is phenethyl, R2 is —H, R3 is phenethyl, and R4 is —H, i.e., the biguanide moiety of the compound is phenformin dimer (which may be termed “Phen²”) (Structure I-F).

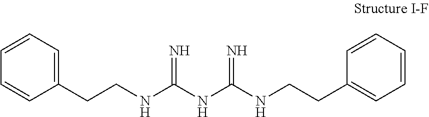 [SMILES: N=C(NCCc1ccccc1)NC(=N)NCCc1ccccc1]

In another particular example, R1 is phenethyl, R2 is —H, R3 is —H, and R4 is -L-Z, wherein L is carboxyl and Z is 1,3,4,6-Tetra-O-acetyl-alpha-D-glucopyranose, i.e., the biguanide moiety of the compound is Structure I-G.

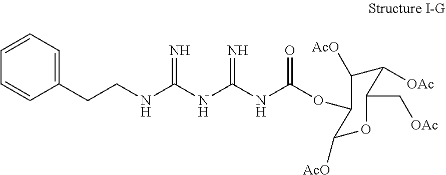 [SMILES: CC(=O)OCC1OC(OC(C)=O)C(OC(=O)NC(=N)NC(=N)NCCc2ccccc2)C(OC(C)=O)C1OC(C)=O]

In one example, M is ^{99m}Tc, R1 is phenethyl, and R2 is —H. Not to be bound by theory, the compound of Formula I of this example can be useful in diagnostic applications, as discussed in more detail below. This compound may be termed “Tc-Phen.”

In one example, M is Re, R1 is phenethyl, and R2 is —H. In particular examples, Re is enriched in the isotope ¹⁸⁶Re, which is a beta particle emitter (t, 3.8 days), ¹⁸⁸Re, which is also a beta particle emitter (t, 17 hours), or both. Not to be bound by theory, the compound of Formula I of this example may be useful in therapeutic applications, as will be discussed in more detail below.

In particular examples, the compound of Formula I may be any of the particular compounds identified above.

### II. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on Formula I include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Exemplary methods for synthesizing the compounds as described herein are provided in the examples below.

### III. Pharmaceutical Formulations

The compounds described herein or derivatives thereof can be provided in a pharmaceutical composition. For example, in addition to the compound of Formula I, the composition may further comprise a pharmaceutically-acceptable carrier. In examples, exemplary pharmaceutically-acceptable carriers include, but are not limited to, carriers selected from the group consisting of polyethoxylated castor oil, ethanol, aqueous saline solutions, any two thereof, and all three thereof. The polyethoxylated castor oil may be that which is commercially available under the trade name Cremophor® (BASF, Ludwigshafen-am-Rhein, Germany).

The composition may further comprise other materials known in the pharmaceutical arts, such as buffers, preservatives, adjuvants, surfactants, diluents (e.g., saline or dextrose) or the like. Such particular other compounds may be routinely selected by the person of ordinary skill in the art having the benefit of the present disclosure. Further details regarding suitable pharmaceutical compositions are detailed below.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS' (BASF; Florham Park, NJ).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, inhalants, and skin patches. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the compounds described herein can be contained in a drug depot. A drug depot comprises a physical structure to facilitate implantation and retention in a desired site (e.g., a synovial joint, a disc space, a spinal canal, abdominal area, a tissue of the patient, etc.). The drug depot can provide an optimal concentration gradient of the compound at a distance of up to about 0.1 cm to about 5 cm from the implant site. A depot, as used herein, includes but is not limited to capsules, microspheres, microparticles, microcapsules, microfibers particles, nanospheres, nanoparticles, coating, matrices, wafers, pills, pellets, emulsions, liposomes, micelles, gels, antibody-compound conjugates, protein-compound conjugates, or other pharmaceutical delivery compositions. Suitable materials for the depot include pharmaceutically acceptable biodegradable materials that are preferably FDA approved or GRAS materials. These materials can be polymeric or non-polymeric, as well as synthetic or naturally occurring, or a combination thereof. The depot can optionally include a drug pump.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.0001 to about 200 mg/kg of body weight of active compound per day, which may be administered in a single dose or in the form of individual divided doses, such as from 1 to 4 times per day. Alternatively, the dosage amount can be from about 0.01 to about 150 mg/kg of body weight of active compound per day, about 0.1 to 100 mg/kg of body weight of active compound per day, about 0.5 to about 75 mg/kg of body weight of active compound per day, about 0.5 to about 50 mg/kg of body weight of active compound per day, about 0.01 to about 50 mg/kg of body weight of active compound per day, about 0.05 to about 25 mg/kg of body weight of active compound per day, about 0.1 to about 25 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, about 5 mg/kg of body weight of active compound per day, about 2.5 mg/kg of body weight of active compound per day, about 1.0 mg/kg of body weight of active compound per day, or about 0.5 mg/kg of body weight of active compound per day, or any range derivable therein. Optionally, the dosage amounts are from about 0.01 mg/kg to about 10 mg/kg of body weight of active compound per day. Optionally, the dosage amount is from about 0.01 mg/kg to about 5 mg/kg. Optionally, the dosage amount is from about 0.01 mg/kg to about 2.5 mg/kg.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

In one example, the composition may further comprise a pharmaceutically-acceptable carrier. Exemplary carriers include those set forth above. The compound of Formula I may be included in the composition in the form of a pharmaceutically-acceptable salt. Appropriate counterions for such salts can be determined by the person of ordinary skill in the art having the benefit of the present disclosure as a routine matter. In examples, the composition may further comprise one or more diagnostic or therapeutic molecules other than the compound of Formula I.

### IV. Methods of Use

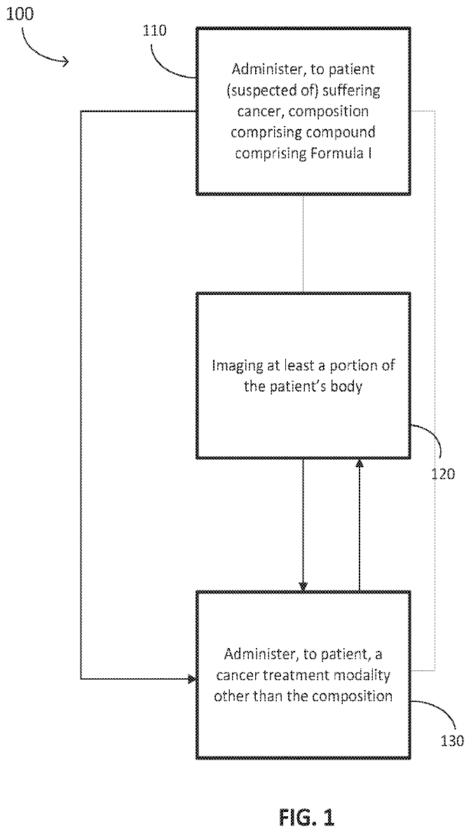

Provided herein are methods to treat a subject having cancer or suspected of having cancer. FIG. 1 presents a flowchart of a method 100 in accordance with examples of the present disclosure. The method 100 comprises administering (at 110), to a patient suffering from a cancer or suspected of suffering from a cancer, a composition comprising a diagnostically-effective amount or a therapeutically-effective amount of a compound as described herein.

By a “diagnostically-effective” amount or a “therapeutically-effective” amount of the compound is meant an amount such that the compound will exert a diagnostic effect or a therapeutic effect after administration. The amount of the compound that will be effective will depend on the particular compound, including its isotope(s) of M, if relevant; the intended application (diagnostic or therapeutic); the diagnostic modality to be used (e.g., CT scan or other imaging technique); the type and subtype of the cancer; the number of doses to be administered in a therapeutic regimen; for certain cancers, the patient's sex, age, and race; the route of administration; and the presence or absence of other compounds in the composition; among other parameters that will be apparent to the person of ordinary skill in the art having the benefit of the present disclosure. Amounts may be measured by mass or, when the method is performed for various diagnostic purposes for which the isotope(s) of M are relevant, by radioactivity.

In one example, wherein M is ^{99m}Tc and the biguanide moiety of the compound is phenformin, the amount of the compound may be from about 400 μCi/kg body weight of the patient to about 100 mCi/kg body weight of the patient.

In one example, wherein M is Re and the biguanide moiety of the compound is phenformin, the amount of the compound may be from about 5 mg/kg body weight of the patient to about 500 mg/kg body weight of the patient.

Alternatively, or in addition, the composition may further comprise a diagnostic or therapeutic compound other than the compound of Formula I.

In the method 100, the patient may be any mammal suffering from or suspected of suffering from the cancer. In one example, the patient is a human being, including pediatric and geriatric populations.

In examples, the present method may be performed in a veterinary context. That is, the patient may be any non-human mammal suffering from or suspected of suffering from a cancer. The non-human mammal may be a research animal, a pet, livestock, a working animal, a racing animal (e.g., a horse, a dog, a camel, etc.), an animal at stud (e.g., a bull, a retired racing stallion, etc.), or any other non-human mammal for which it is desired to treat its cancer.

For convenience, the description will typically refer to human patients. However, the person of ordinary skill in the art having the benefit of the present disclosure will readily be able to adapt the teachings of the present disclosure to a veterinary context.

By “suffering from a cancer” is meant that the cancer is detectable in the patient's body using any diagnostic technique presently known or to be discovered. “Suffering” does not require the patient to be in pain from or have any naturally-perceptible symptoms of the cancer. Generally, as is known, the earlier a cancer can be treated, including before the patient notices pain or any other symptoms, the greater the chances of remission. By “suspected of suffering from a cancer” is meant that a medical professional considers it possible, based on one or more symptoms presented by a patient and/or a prior history of cancer of the patient, that the patient may suffer from a cancer which has not yet been detected. As should be apparent, a patient “suspected of suffering from a cancer” may be cancer-free and/or in remission from a prior suffering of cancer.

The present method may be used to treat any type of cancer. In one example, the cancer is pancreatic cancer. In some cases, cancers contemplated for treatment using the methods described herein include solid tumors such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, non-small cell lung cancer, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma. Cancers also include blood-borne cancers, such as acute lymphoblastic leukemia (ALL), acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, and multiple myeloma. Cancer also includes acute and chronic leukemias such as lymphoblastic, myelogenous, lymphocytic, and myelocytic leukemias. Cancer also includes lymphomas such as Hodgkin's disease, non-Hodgkin's Lymphoma, Waldenstrom's macroglobulinemia, heavy chain disease, and poly cythemia vera.

The composition may be administered (at 110) to the patient by any route. Such routes may be characterized as systemic or local. Systemic routes include oral, nasal, buccal, intraperitoneal, and intravenous injection routes, among others. Local routes include subcutaneous, intramuscular, intraorganal, and intratumoral injection, and catheterized and endoscopic routes, among others. Generally, local routes in proximity to malignant cells of the cancer are desirable, in that they may require lower doses of the composition and may reduce the risk of side effects. However, the person of ordinary skill in the art can, with the application of routine skill and the benefit of the present disclosure, use systemic delivery routes.

In the method 100, administering (at 110) the composition may be performed in a single dose or a plurality of doses. If a plurality of doses is performed, the number of doses and the time between doses can be selected as a routine matter by the person of ordinary skill in the art having the benefit of the present disclosure.

The method 100 may include one or more additional events. In one example, the method 100 further includes imaging (at 120) at least a portion of the patient's body after the administering. In particular examples in which the method 100 comprises imaging (at 120), M in the compound of Formula I may be ^{99m}Tc. In a particular example, M is ^{99m}Tc, R1 is phenethyl, and R2 is —H.

Any desired imaging modality may be used when imaging (at 120). In one example, the imaging (at 120) comprises computed tomography (CT) scanning. Known techniques for CT scanning may be used in a routine matter by the person of ordinary skill in the art having the benefit of the present disclosure.

Alternatively, or in addition, the method 100 may further comprise administering (at 130), to the patient, a cancer treatment modality other than the compound of Formula I. A wide variety of cancer treatment modalities other than the compound of Formula I are known to the person of ordinary skill in the art and need not be described in detail here. By way of example, in one example, the cancer treatment modality other than the compound of Formula I is selected from the group consisting of surgical resection, chemotherapy with a compound other than the compound of Formula I, immunotherapy, checkpoint inhibitor therapy, oncolytic virus therapy, thermal therapy (e.g., RFA, microwave ablation, and/or cryotherapy), radiotherapy, and two or more thereof.

Optionally, an additional therapeutic agent can be administered, such as a chemotherapeutic agent. A chemotherapeutic agent is a compound or composition effective in inhibiting or arresting the growth of an abnormally growing cell. Thus, such an agent may be used therapeutically to treat cancer as well as other diseases marked by abnormal cell growth. Illustrative examples of chemotherapeutic compounds include, but are not limited to, bexarotene, gefitinib, erlotinib, gemcitabine, paclitaxel, docetaxel, topotecan, irinotecan, temozolomide, carmustine, vinorelbine, capecitabine, leucovorin, oxaliplatin, bevacizumab, cetuximab, panitumumab, bortezomib, oblimersen, hexamethylmelamine, ifosfamide, CPT-11, deflunomide, cycloheximide, dicarbazine, asparaginase, mitotant, vinblastine sulfate, carboplatin, colchicine, etoposide, melphalan, 6-mercaptopurine, teniposide, vinblastine, antibiotic derivatives (e.g. anthracyclines such as doxorubicin, liposomal doxorubicin, and diethylstilbestrol doxorubicin, bleomycin, daunorubicin, and dactinomycin); antiestrogens (e.g., tamoxifen); antimetabolites (e.g., fluorouracil (FU), 5-FU, methotrexate, floxuridine, interferon alpha-2B, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cisplatin, vincristine and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chlorambucil, mechlorethamine (nitrogen mustard) and thiotepa); and steroids (e.g., bethamethasone sodium phosphate).

Any of the aforementioned therapeutic agents can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

Administering (at 130) the cancer treatment modality other than the compound of Formula I may be targeted against the same cancer as the composition, against metastases thereof, against a primary tumor or metastases of a cancer other than cancer targeted by the composition, or two or more thereof.

Regardless of the particular cancer treatment modality other than the compound of Formula I, if one or more is/are administered (at 130), the administering (at 130) may be performed before, after, or simultaneously with the administering (at 120) the composition comprising the compound of Formula I. Particular relative and absolute timing of administering (at 110) the composition comprising the compound of Formula I and administering (at 130) the other cancer treatment modality or modalities will be a routine matter for the person of ordinary skill in the art having the benefit of the present disclosure. For example, administering (at 130) may occur after, before, or simultaneously with administering (at 120).

The methods and compounds as described herein are useful for both prophylactic and therapeutic treatment. For prophylactic use, a therapeutically effective amount of the compounds and compositions or pharmaceutically acceptable salts thereof as described herein are administered to a subject prior to onset (e.g., before obvious signs of a tumor development), during early onset (e.g., upon initial signs and symptoms of tumor development), or after the development of a tumor. Prophylactic administration can occur for several days to years prior to the manifestation of symptoms of cancer. Therapeutic treatment involves administering to a subject a therapeutically effective amount of the compounds and compositions or pharmaceutically acceptable salts thereof as described herein after a cancer is diagnosed.

The methods herein for prophylactic and therapeutic treatment optionally comprise selecting a subject with or at risk of developing cancer. A skilled artisan can make such a determination using, for example, a variety of prognostic and diagnostic methods, including, for example, a personal or family history of the disease or condition, clinical tests (e.g., imaging, biopsy, genetic tests), and the like. Optionally, the methods herein can be used for preventing relapse of cancer in a subject in remission (e.g., a subject that previously had cancer).

### V. Kits

Also provided herein are kits for treating or preventing a cancer in a subject. A kit can include any of the compounds or compositions described herein. For example, a kit can include one or more compounds of Formula I. Optionally, the kit can include a diagnostically-effective amount or a therapeutically-effective amount of a compound of Formula I. The kit can also include instructions for use of the composition in a method comprising administering, to a patient suffering from a cancer or suspected of suffering from a cancer, the composition.

A “kit,” as used herein, refers to a package containing the composition, and instructions of any form that are provided in connection with the composition in a manner such that a clinical professional will clearly recognize that the instructions are to be associated with the composition. “Instructions” typically involve written text or graphics on or associated with packaging of compositions as described herein. Instructions also can include any oral or electronic instructions provided in any manner. Written text or graphics may include a website URL or a QR code encoding a website URL, where other instructions or supplemental information may be provided in electronic form.

The kit may contain one or more containers, which can contain the composition or a component thereof. The kits also may contain instructions for mixing, diluting, or administering the composition. The kits also can include other containers with one or more solvents, surfactants, preservatives, and/or diluents (e.g., normal saline (0.9% NaCl), or 5% dextrose) as well as containers for mixing, diluting, or administering the composition to the patient in need of such treatment.

The composition may be provided in any suitable form, for example, as a liquid solution or as a dried material. When the composition provided is a dry material, the material may be reconstituted by the addition of solvent, which may also be provided by the kit. In examples where liquid forms of the composition are used, the liquid form may be concentrated or ready to use. The kit, in one example, may comprise a carrier being compartmentalized to receive in close confinement one or more containers such as vials, tubes, and the like.

The composition is described above. In one example, in the compound of Formula I, M is ^{99m}Tc, R1 is phenethyl, and R2 is —H. In another example, in the compound of Formula I, M is Re, R1 is phenethyl, and R2 is —H.

The method is described above. In one example, the instructions further comprise instructions to image at least a portion of the patient's body after the administering. In one further example of a kit according to this example, M may be ^{99m}Tc.

A kit including instructions to image may include two compositions, each comprising a compound of Formula I, with a first composition comprising a compound wherein M is ^{99m}Tc, wherein the instructions call for using the first composition for imaging; and a second composition comprising a compound wherein M is Re, wherein the instructions call for using the second composition for treatment of a cancer.

Alternatively, or in addition, the instructions of the kit may further comprise instructions to administer, to the patient, a cancer treatment modality selected from the group consisting of surgical resection, chemotherapy with a compound other than the compound of Formula I, immunotherapy, checkpoint inhibitor therapy, oncolytic virus therapy, thermal therapy, radiotherapy, and two or more thereof. In examples, such a kit may further comprise one or more of a chemotherapeutic compound other than the compound of Formula I, a cancer vaccine reagent, a checkpoint inhibitor, a monoclonal antibody, an oncolytic virus, or two or more thereof.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs (e.g., size of the tumor or rate of tumor growth) of the disease in a subject as compared to a control. As used herein, control refers to the untreated condition (e.g., the tumor cells not treated with the compounds and compositions described herein). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

The following examples are included to demonstrate certain aspects of the disclosure, and are not intended to limit the scope of the claims. It should be appreciated by those of skill in the art that the techniques disclosed in the examples which follow represent techniques discovered as detailed herein to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### EXAMPLES

### Example 1: Metal-Based Compounds for Use in Cancer Theranostics

The following examples disclose a novel combination of mitotropic agents (biguanides) and metal ions to achieve real time visualization of tumor uptake and biodistribution (with ^{99m}Tc complexes) and with increased therapeutic effects (with Re complexes). The technology can be further applied to therapeutic radionuclides of Re (e.g., ¹⁸⁸Re, a beta emitter) as well. These metal-based compounds can be used for the purpose of both diagnosis and therapy (theranositics).

Metformin (N,N-dimethyl biguanide) is a biguanide drug commonly used in the treatment of type 2 diabetes. Epidemiological studies have shown that metformin use is related to decreased incidence of pancreatic cancer in diabetic patients. See Evans J M, et al. Metformin and reduced risk of cancer in diabetic patients. BMJ. 2005; 330: 1304-1305; see also Heckman-Stoddard B M, et al. Repurposing old drugs to chemoprevention: the case of metformin. Semin Oncol. 2016; 43: 123-133. Metformin and its analogue phenformin (N-phenylethyl biguanide) display antitumor effect in multiple types of cancer, including pancreatic cancer. Hirsch H A, et al. Metformin selectively targets cancer stem cells, and acts together with chemotherapy to block tumor growth and prolong remission. Cancer Res. 2009; 69: 7507-7511; Appleyard M V C L, et al. Phenformin as prophylaxis and therapy in breast cancer xenografts. Brit J Cancer. 2012; 106: 1117-1122; Rajeshkumar N V, et al. Treatment of pancreatic cancer patient-derived xenograft panel with metabolic inhibitors reveals efficacy of phenformin. Clin Cancer Res. 2017; 23: 5639-5647. The mechanism is believed to involve activation of the AMP-activated protein kinase (AMPK), resulting in vulnerability to an energy crisis. The antitumor effects of biguanides are believed to result in part from their inhibition of complex I in mitochondrial electron transfer chain and their actions on oxidative phosphorylation. Bridges Hannah R, et al. Effects of metformin and other biguanides on oxidative phosphorylation in mitochondria. Biochem J. 2014; 462: 475-487; Foretz M, et al. Metformin: from mechanisms of action to therapies. Cell Metab. 2014; 20: 953-966; Wheaton W W, et al. Metformin inhibits mitochondrial complex I of cancer cells to reduce tumorigenesis. Elife. 2014; 3: e02242. Many tumors rely on oxidative phosphorylation for bioenergetic and biosynthetic processes (e.g., aspartate synthesis). Birsoy K, et al. An essential role of the mitochondrial electron transport chain in cell proliferation is to enable aspartate synthesis. Cell. 2015; 162: 540-551; Viale A, et al. Oncogene ablation-resistant pancreatic cancer cells depend on mitochondrial function. Nature. 2014; 514: 628-632; Molina J R, et al. An inhibitor of oxidative phosphorylation exploits cancer vulnerability. Nat Med. 2018; 24: 1036-1046. The gamma particle emitter technetium-99m (^{99m}Tc, t½, =6 h, Eγ=141 keV) is widely used for scintigraphy and single-photon emission computed tomography (SPECT) in nuclear medicine. Both ^{99m}Tc and rhenium (Re) are group VIIB transition metal elements. Because of chemical similarities, the nonradioactive isotopes of Re have been used as a surrogate for ^{99m}Tc to elucidate structures and mechanisms. Alberto R. New organometallic technetium complexes for radiopharmaceutical imaging. In: Krause W, editor. Contrast Agents III: Springer Berlin Heidelberg, 2005:1-44. Therefore, structurally matched ^{99m}Tc-labeled and Re-labeled tumor-homing ligands are promising theranostic pairs. Data for the prototype theranostic pair, designated as (M-Phenformin or M-Phen, where M=^{99m}Tc, Re), demonstrated activity in altering mitochondrial function and metabolic state, and showed in vivo antitumor activity as a single agent.

Importantly, the biguanide structures are nitrogen bidentate donors capable of coordinating transition elements. Electronic-structural studies on biguanide indicate that C2-N4-C5-N6 4 pi-electron conjugation is the basic feature of global minimum tautomer for this class of compounds. See Bharatam P V et al., Pharmacophoric features of biguanide derivatives: An electronic and structural analysis. J Med Chem. 2005; 48: 7615-7622. This characteristic makes the electrons delocalized in that region, allowing biguanides to be used as bidentate or tridentate donors to form complexes in the presence of CO. The M(CO)₃-biguanide complexes therefore serve both as a pharmacophore and a radio-ligand for cancer theranostic applications.

### Example 2: Synthesis of the Re-Biguanide Complexes

The Re-biguanide complexes were synthesized by adding a stoichiometric amount of (NEt₄)₂[Re(CO)₃Br₃] in methanol to an aqueous solution of metformin or phenformin. The reaction mixture was stirred overnight and purified by preparative high-performance liquid chromatography (HPLC).

### Example 3: Synthesis of the ^{99m}Tc-Biguanide Complexes

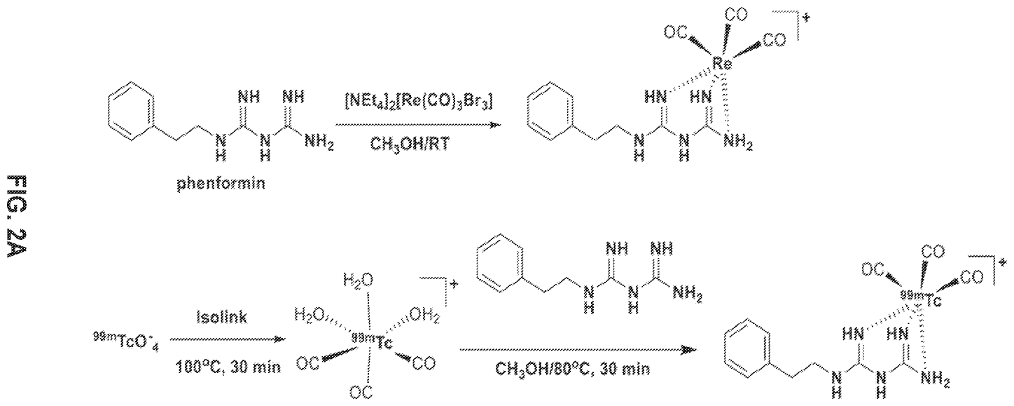

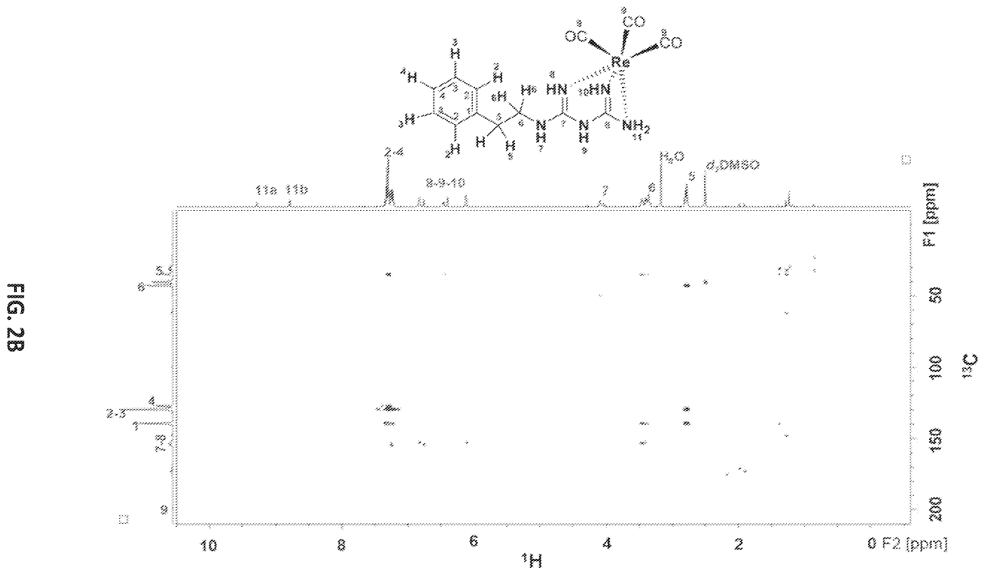

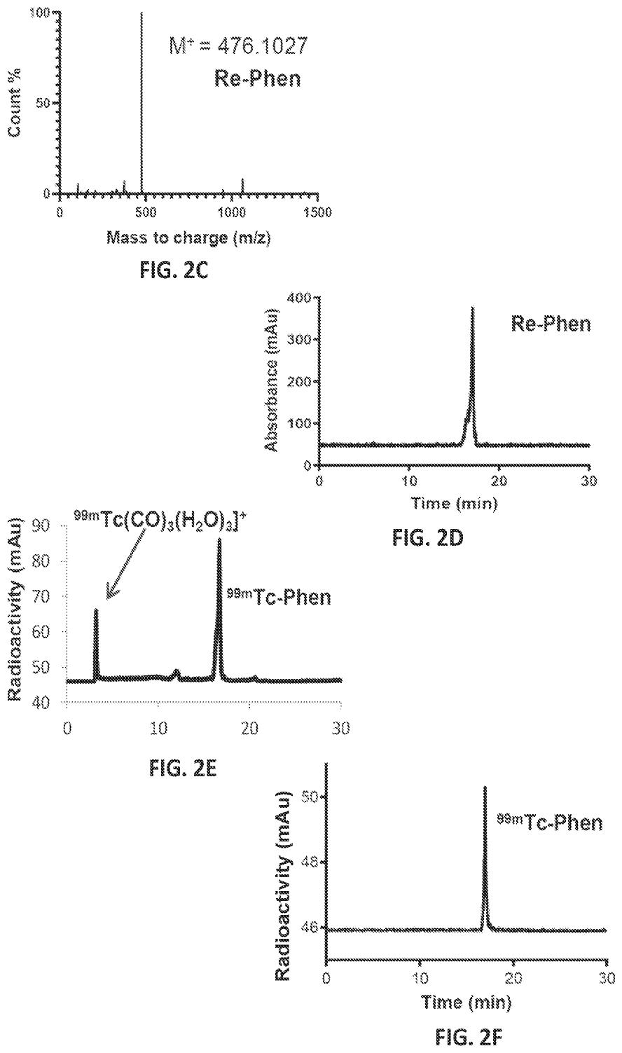

^{99m}Tc-biguanide complexes were synthesized from ^{99m}Tc-tricarbonyl aqua ion ^{99m}Tc(CO)₃(H₂O)₃]⁺. ^{99m}Tc(CO)₃(H₂O)₃]⁺ was prepared using the following general procedure: 1.0 mL of ^{99m}TcO4-(2-5 mCi) was added to an Isolink carbonyl kit vial containing sodium tartrate Na₂C₄H₄O₆, sodium tetraborate Na₂B₄O₇, sodium carbonate Na₂CO₃, and sodium boranocarbonate Na₂H₃BCO₂. The solution was heated in water bath at 100° C. for 20 min. The solution was then cooled for 5 min and vented. The resulting solution was neutralized with 100 μL of 1 M HCl to pH approximately 6-7 to decompose residual boranocarbonate. [^{99m}Tc(CO)₃(H₂O)₃]⁺ (0.5 mL, 2 mCi) was then added to a sealed vial containing 10-20 μg of metformin or phenformin in distilled water. Synthetic scheme for Re-Phen and ^{99m}Tc-Phen are presented in FIG. 2A.

### Example 4: Characterization of the Re-Biguanide and ^{99m}Tc Complexes

The resulting compounds were characterized by NMR, high resolution LC-MS, and analytical HPLC. The NMR HMBC (Heteronuclear Multiple Bond Correlation) experiment gives correlations between carbons and protons that are separated by two, three, and, sometimes in conjugated systems, four bonds, where the protons lie along the observed F2 (X) axis and the carbons are along the F1 (Y) axis. The spectrum of Re-Phen at 500 MHz is shown in FIG. 1B. The peaks of carbons are assigned in red numbers and the peaks of protons assigned in blue numbers. The cross peaks represent the correlation between carbon and proton through two-bond or three-bond coupling. HPLC-MS was performed on an Agilent LC-MS system (Santa Clara, CA) in the positive ion mode using the electrospray ionization method. Re-Phen was found to have m/e value of 476.1027 (calc. C₁₃H₁₅N₅O₃Re, 476.072) by high resolution mass spectroscopy-electron spray ionization (HRMS-ESI) (FIG. 2C), consistent with the proposed structures of Re-Phen (FIG. 2A). Re-metformin was found to have m/e value of 400.0405 (calc. C₇H₁₁N₅O₃Re, 400.0419), also consistent with its proposed structure.

For analytical HPLC, a Vydac 10×150 mm C18 peptide/protein column was employed using a gradient of 0.05M triethylamine phosphate buffer (solvent A) and methanol (solvent B), with 10-90% solvent A over a period of 30 min at a flow rate of 10 mL/min. The HPLC retention time for Re-Phen was 16.8 min under these conditions (FIG. 2D). Because of the hydrophobic nature of the Re(CO)₃ core moiety, Re-Phen was more lipophilic than phenformin. The log P values for phenformin and Re-Phen measured from octanol and phosphate-buffered saline (PBS) at pH 7.4 were −0.72±0.02 and 1.07±0.08, respectively.

### Example 5: Radiolabeling of Phenformin with ^{99m}Tc and Re

The radiolabeling of phenformin with the [^{99m}Tc(CO)₃(H₂O)₃]⁺ precursor was performed in neutral aqueous PBS buffer solution (pH 7). ^{99m}Tc-Phen was obtained with excellent radiochemical purity (>95%) after HPLC purification. [^{99m}Tc(CO)₃(H₂O)₃]⁺ and excess of unlabeled phenformin could be readily separated from ^{99m}Tc-Phen as ^{99m}Tc-Phen is more lipophilic than both species. Representative chromatograms of Re-Phen before and after purification are shown in FIG. 1E.

### Example 6: Noninvasive Tumor Imaging with ^{99m}Tc-Phen

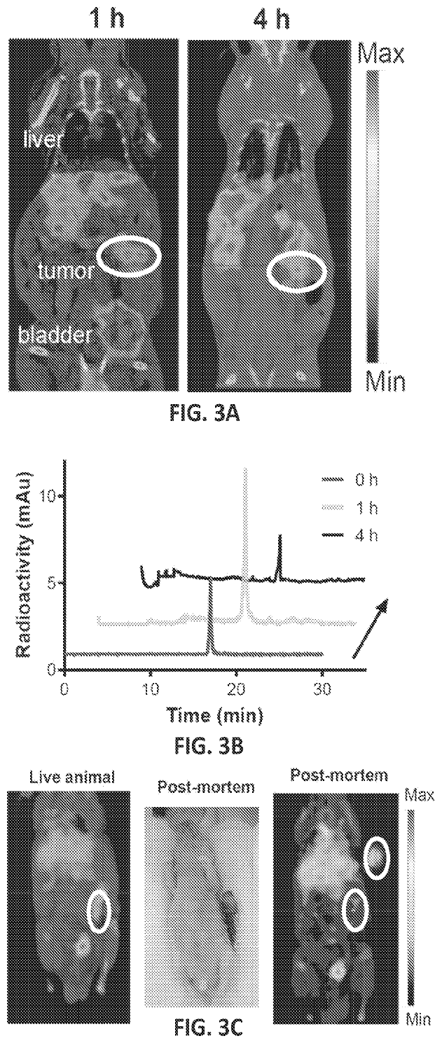

One advantage of Re-Phen is the use of radioactive iso-structure counterpart ^{99m}Tc-Phen to determine biodistribution and level of tumor targeting. Therefore, a tumor-imaging study was performed on orthotopic MiaPaca-2 bearing nude mice following intravenous injection. For μSPECT/CT, mice bearing orthotopic Miapaca-2 tumors (n=4) were anesthetized with isoflurane (2% in oxygen) and placed in a prone position after intravenous (IV) injection of 200-250 μCi ^{99m}Tc-Phen in 0.1 mL of saline. Images were acquired for 20 min using an Albira micro SPECT/PET/CT system (Bruker, Billerica, MA). SPECT system parameter were as follows: two CsI(Na) single crystal gamma cameras, single and multi-pinhole collimator, fully automated field-of-view (FOV) selection. The CT imaging parameters were as follows: 35 μm or smaller voxel sizes, X-ray source 10-50 kVp with 35 μm X-ray spot size, two-dimensional 12 cm×12 cm, 2400×2400 pixel detector. Images were reconstructed using Albira software suite. SPECT and CT image fusion and image analysis were performed using Bruker integrated PMOD and Volview Workplace. MicroSPECT/CT of pancreas with tumors were clearly visible at 1 hour and 4 hours after injection of ^{99m}Tc-Phen (FIG. 3A). SPECT/CT also revealed that radioactivity was washed out through the kidneys into the bladder, and that the radiotracer had a relatively high uptake in the liver (FIG. 3A). Because the radioactive ^{99m}Tc-associated species was excreted into the urine as early as 1 hour post-injection, the metabolic stability of ^{99m}Tc-Phen was examined by analyzing the urine samples collected from mice at different time intervals after a bolus injection of ^{99m}Tc-Phen. The elution times of ^{99m}Tc-Phen before injection and radioactive species in urine samples collected at different time points after intravenous injection of ^{99m}Tc-Phen were compared. Results show that majority of injected ^{99m}Tc-Phen was excreted intact at both 1 hour and 4 hour time points (FIG. 3B), confirming excellent in vivo stability. The Post-mortem images were acquired after removal of pancreas/tumors and the data confirmed imaging findings in live animals (FIG. 3C).

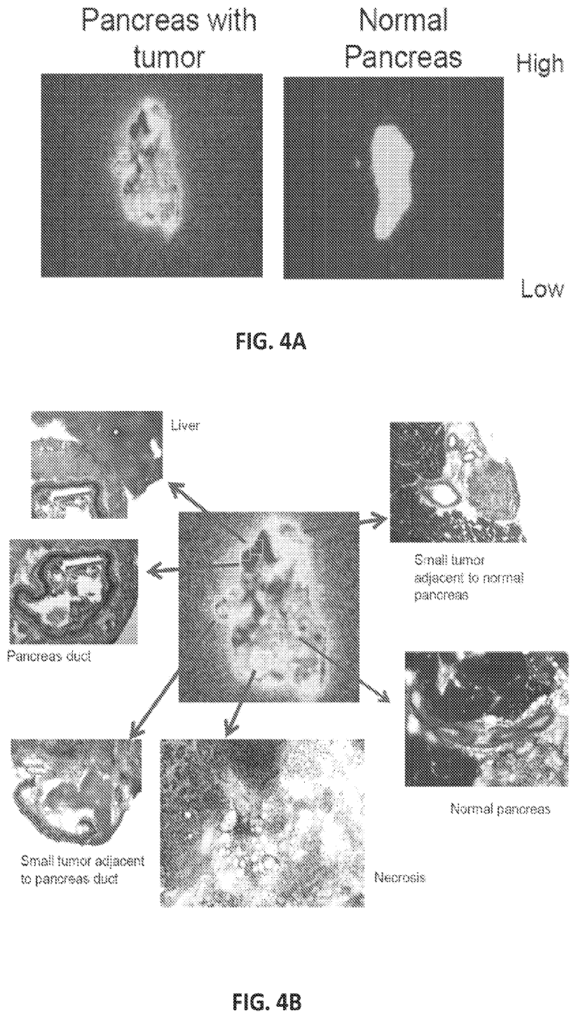

To further confirm tumor uptake of ^{99m}Tc-Phen, the pancreas from tumor-bearing mice and healthy mice collected at 1 hour post-injection were cryosectioned (thickness, 10 μm), and the slices were dried at 40° C. in open air. The sections were then photographed and exposed on BAS-SR 2025 Fuji phosphorous film, and the film was scanned with FLA5100 Multifunctional Imaging System (Fujifilm Medical Systems USA, Stamford, CT). Adjacent slice from each tumor was stained with hematoxylin and eosin (H&E). The cell nuclei were counterstained with 4′,6-diamidino-2-phenylindole (DAPI, Sigma). Compared to pancreas from healthy mice, pancreas from tumor-bearing mice showed markedly higher radioactivity throughout the organ (FIG. 4A). The intra-pancreas distribution of radiotracer in pancreas from the tumor-bearing mouse was heterogeneous, with hot spots corresponding to tumor and peritumoral areas, and relatively colder spots corresponding to pancreatic or necrotic tissues (FIG. 4B).

### Example 7: Biodistribution of ^{99m}Tc-Phen in Treated Mice

For the biodistribution study, radiotracer (200 μCi) was injected IV, and groups of animals were euthanized at 1 hour and 4 hours after injection. Healthy mice without tumor were used as controls. Tumors and other major organs were collected, weighted, and radioactivity counted by using a Cobra gamma counter (Packard). The intestines was emptied of food content prior to radioactivity measurement. The percentage of injected dose per gram of tissue (% ID/g) was calculated by dividing the % ID/tissue by the weight of the tissue.

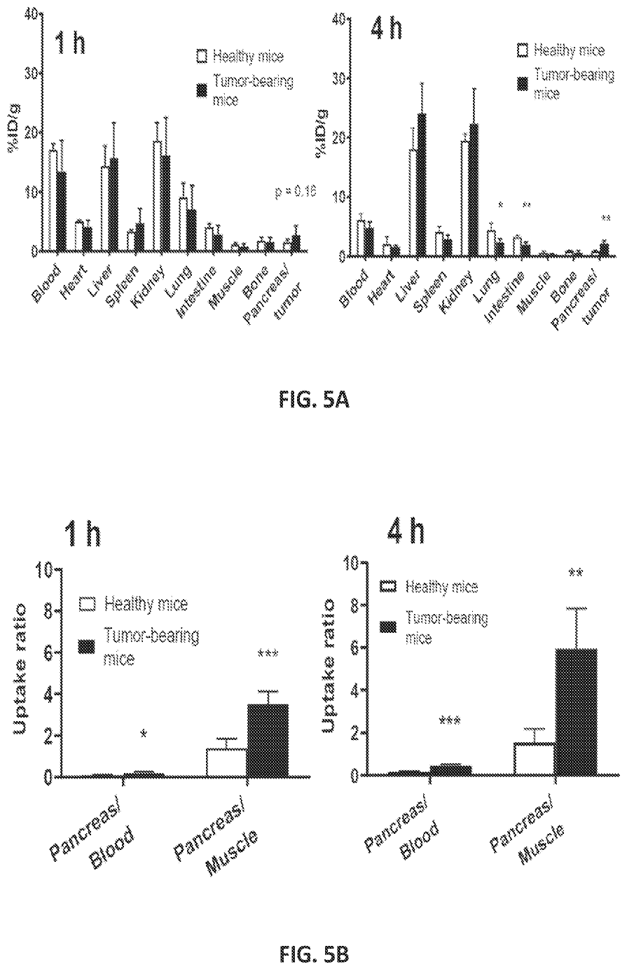

The biodistribution data were consistent with imaging results. At 1 hour after IV injection of ^{99m}Tc-Phen, blood, liver, and kidney had relatively high radioactivity in both healthy and tumor-bearing nude mice. Pancreas uptake of the radiotracer was moderately higher in tumor-bearing mice than in healthy mice (p=0.016). At 4 hours post-injection, blood activity was reduced compared to that at 1 hour post-injection, but radioactivities in the liver and the kidney were largely retained. In healthy mice and tumor-bearing mice, the uptakes of ^{99m}Tc-Phen at 4 hours post-injection in the blood (6.14±1.02% ID/g vs. 4.81±1.00% ID/g; p=0.11), liver (18.0±3.6% ID/g vs. 24.0±5.1% ID/g; p=0.10), and kidney (19.5±1.1% ID/g vs. 22.3±5.9% ID/g, p=0.38) had no significant differences. However, ^{99m}Tc-Phen was found to have significantly higher uptake in the pancreas of tumor-bearing mice (2.19±0.45% ID/g) than in the pancreas of healthy mice (0.94±0.10% ID/g; p=0.0016) (FIG. 5A). At both 1 hour and 4 hours post-injection, the pancreas-to-blood and pancreas-to-muscle ratios in the tumor-bearing mice were significantly higher than in the healthy mice (FIG. 5B). Increasing tumor uptake over time suggest tumor-specific retention of ^{99m}Tc-Phen.

### Example 8: Re-Phen Display Single-Agent Antitumor Activity Against PDAC

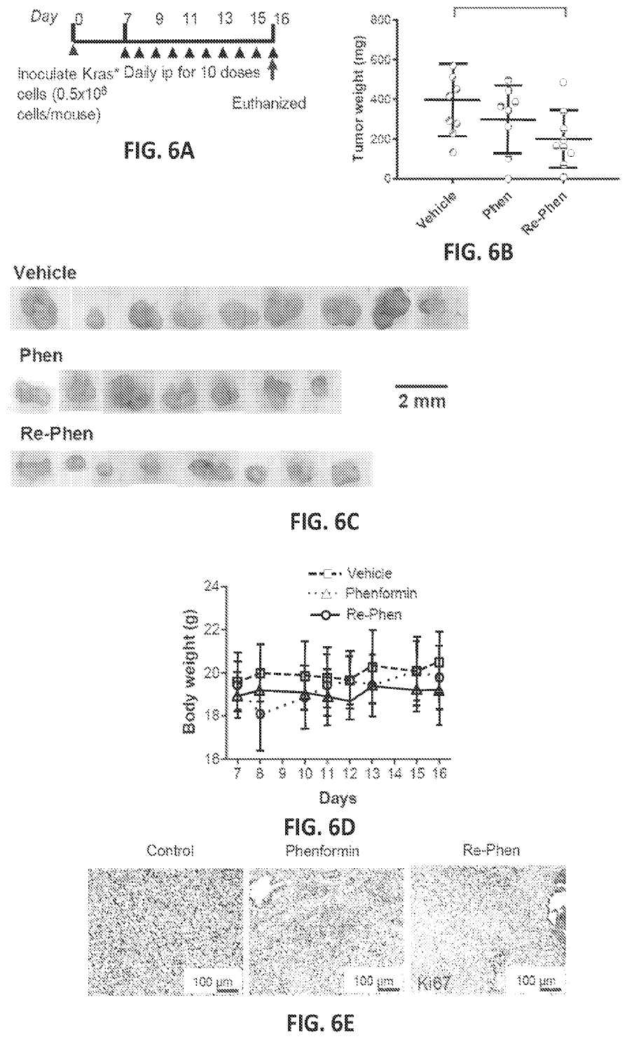

For antitumor efficacy study, the syngeneic Kras* orthotopic PDAC model was used. Kras* PDAC cells established from transgenic mouse model of PDAC with inducible KrasG12D mutation were inoculated into the pancreas of 6-week-old female C57BL/6 mice (Taconic Biosciences, Rensselaer, NY). Treatments were initiated on day 7 after tumor cells inoculation when tumors were ˜5 mm in diameter. Each mouse received 10 daily intraperitoneal injections of phenformin or Re-Phen at a dose of 50 mg/kg/injection (100 μL/mouse) (FIG. 6A). Each drug was formulated in 50/50 (v/v) Cremorphor EL/absolute ethanol, which that was further diluted at 1:4 (v/v) ratio with saline before injection. Animals were weighted daily to record possible adverse effects. Animals were euthanized on day 16 after the last injection. Tumors were harvested and weighted, fixed in formalin, embedded in paraffin, and cut into 4-μm sections. Slides were immunohistochemically stained with the proliferation marker Ki67, counterstained with hematoxylin and visualized under a brightfield microscope at 100× or 200× magnification.

Treatments with Re-Phen but not phenformin significantly reduced tumor size compared to vehicle control (FIG. 6B, 5C). No significant change in body weight was noted for both phenformin and Re-Phen treated mice (FIG. 6D). IHC showed markedly reduced Ki67+ proliferating cells in tumors of mice treated with phenformin and Re-Phen compared to vehicle-treated mice (FIG. 6E). These data indicate that Re-Phen displayed single agent antitumor activity in the animal model tested.

### Example 9: Re-Phen Disrupted Mitochondrial Functions

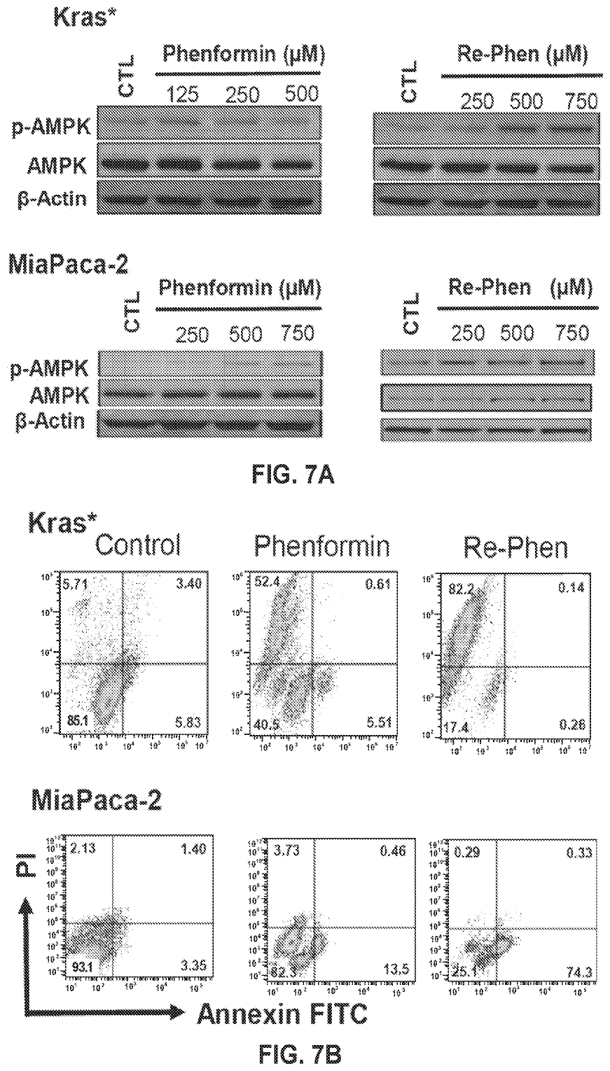

Both phenformin and Re-Phen activated AMPK (FIG. 7A). AMPK is a key enzyme that becomes activated under nutrient stress. Annexin V/PI double staining kit was used to detected cell death in flow cytofluorimetric analyses. The data indicated that the mode of cell death depended on cell line used. In Kras* cell line, cells died largely of necrosis; in MiaPaca-2 cell line, cells died mostly of apoptosis. In both cases, Re-Phen was more potent than phenformin in inducing cell death (FIG. 7B).

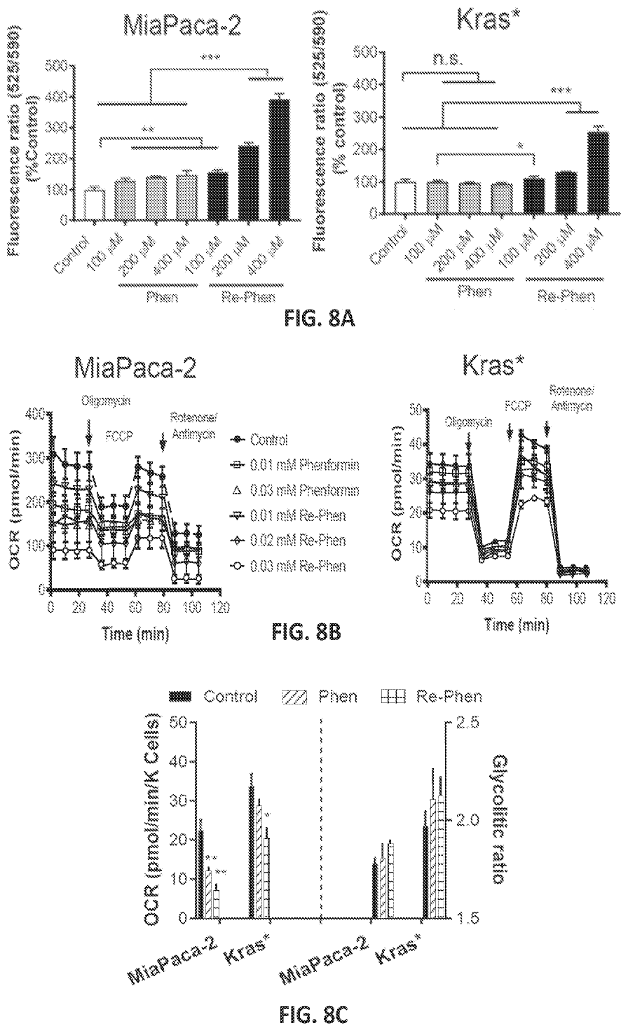

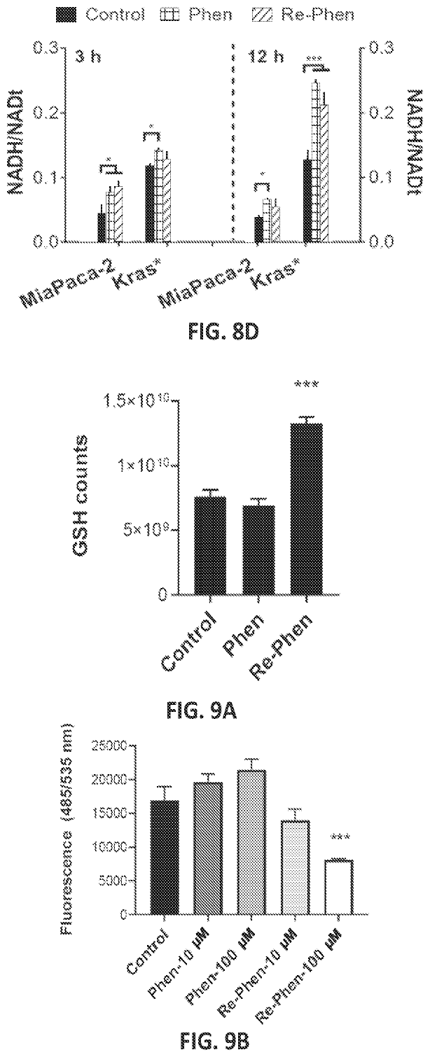

Both Re-Phen and phenformin altered the mitochondrial functions and metabolic state of PDAC cells. The fluorescent intensities for both monomeric forms (Ex/Em=490/525 nm) and J-aggregates (Ex/Em=540/590 nm) of JC-10 change upon mitochondrial membrane polarization. Thus, an increase in the ratio of fluorescence intensities measured at 525 nm (monomeric form) over 590 nm (aggregates) represents a decrease in mitochondrial membrane potential. Treatments with both compounds reduced mitochondrial membrane potential as measured by using the JC-10 microplate assay kit (Abcam, Cambridge, MA) (FIG. 8A). Both Re-Phen and phenformin suppressed mitochondria respiration (FIG. 8B), and increased compensatory glycolysis reflected by an increase in glycolic ratio, which is the ratio of lactate produced to glucose consumed (FIG. 8C). Treatments with phenformin and Re-Phen also increased the ratio of NADH to total NADt ((NAD+NADH) (FIG. 8D). These data suggest that similar to phenformin, Re-Phen acted as a complex I inhibitor and disrupted mitochondrial functions in PDAC cells.

### Example 10: Re-Phen Impacted the Redox State of PDAC Cells

Using mass spectrometry-based metabolomics analysis, it was determined that Re-Phen but not phenformin treatments increased the level of glutathione (GSH) in MiaPaca-2 cells (FIG. 9A). Re-Phen also reduced the level of reactive oxygen species (ROS) whereas phenformin increased the level of ROS (FIG. 9B). These data suggest that Re-Phen and phenformin induced different effects on redox state.

### Example 11: Re-Phen Changed Metabolomics of PDAC Cells

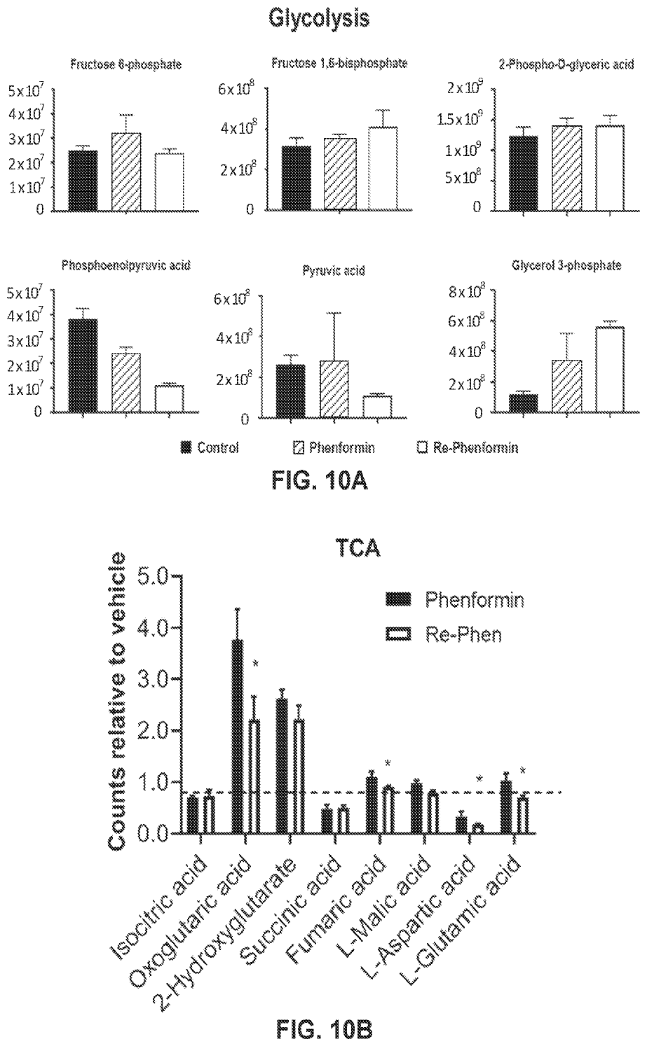

To investigate impact of R-Phen treatment on metabolic activity of PDAC cells, MiaPaca-2 cells were treated with Re-Phen or phenformin at 100 μM for 12 h. Following, untargeted IC-MS was used to analyze changes in cellular metabolites. It was determined that Re-Phen suppressed the level of phosphoenolpyruvate (PEP) but increased the level of 3-phosphoglycerate (3-PG) more than phenformin did (FIG. 10A).

Accumulation of NADH may inhibit a number of dehydrogenases in the TCA cycle including pyruvate dehydrogenase, isocitrate dehydrogenase, and 2-oxoglutarate dehydrogenase (OGDH). Both phenformin and Re-Phen suppressed TCA cycle as reflected by reduced level of succinate and aspartate in drug-treated cells compared to vehicle-treated control cells (FIG. 10B). The observed increase in the levels of oxoglutarate and hydroxyglutarate may be attributed to increased glutaminolysis to compensate for the TCA cycle suppression and for the needs of macromolecule biosynthesis. Decreased level of aspartate by phenformin in ovarian cancer cells has been reported by others. Together these experiments suggest the enzymes regulating PEP and aspartate metabolism in cells are involved in phenformin and Re-Phen's inhibitory activity against PDAC cells.

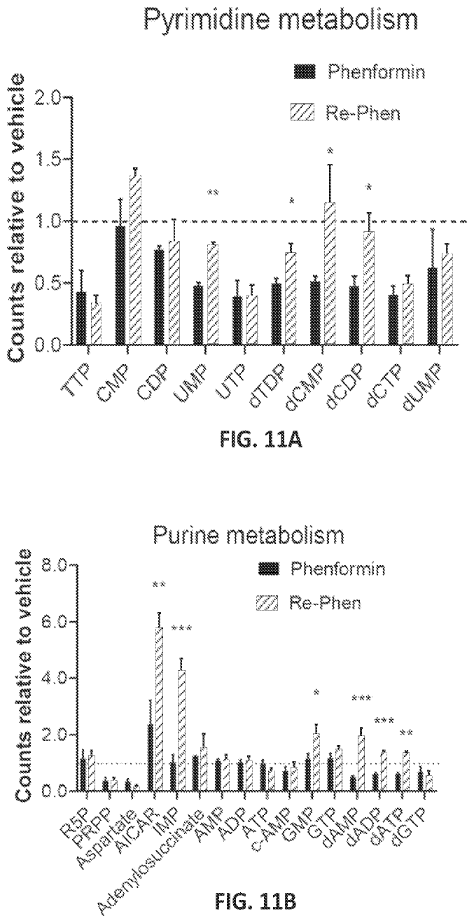

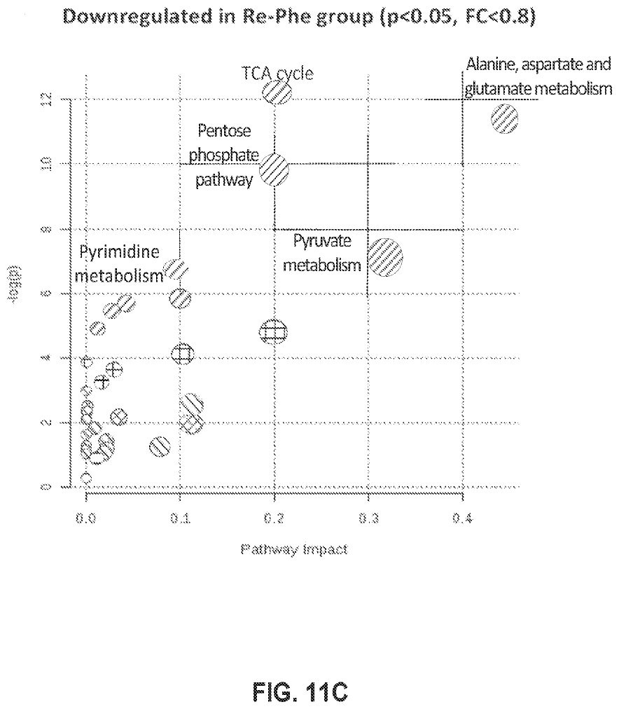

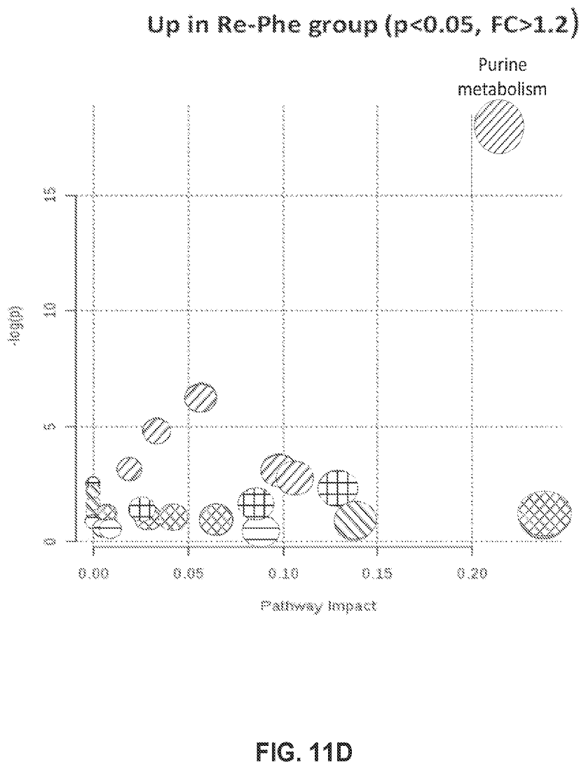

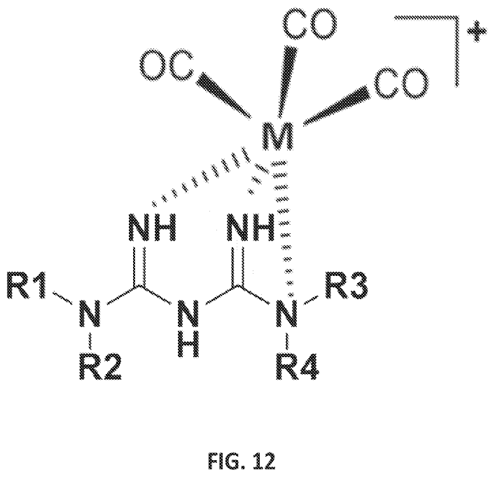

Both phenformin and Re-Phen decreased pyrimidine nucleotide pool (FIG. 11A). Surprisingly, compared to phenformin, Re-Phen increased purine nucleotide pool (FIG. 11B). Pathway analysis showed that compared to phenformin, Re-Phen downregulated glycolysis, TCA, pentose phosphate pathway (PPP), and pyrimidine biosynthesis pathway, while Re-Phen promoted purine biosynthesis (FIG. 11C). These data suggest that Re-Phen and phenformin displayed different effects on de novo nucleotide synthesis. In summary, Re-Phen altered the metabolic programming and redox state of PDAC cells in a way different from that of phenformin.

In summary, the data suggest that decrease in the mitochondrial respiration rate and alteration of redox state are possible mechanisms of anticancer effects of Re-biguanides, specifically Re-Phen. Mitochondria-targeted compounds may induce a novel redox-signaling mechanism in which reduced ROS and increased glutathione play a critical role in the antiproliferative effects. Sullivan L B & Chandel N S. Mitochondrial reactive oxygen species and cancer. Cancer Metab. 2014; 2: 17; Diebold L & Chandel N S. Mitochondrial ROS regulation of proliferating cells. Free Radical Biology & Medicine. 2016; 100: 86-93. The hyperpolarized mitochondrial membrane of cancer cells is a shared feature of many types of cancer, including PDAC, and represents a therapeutic vulnerability for cancer cells. Zorova L D, et al. Mitochondrial membrane potential. Anal Biochem. 2018; 552: 50-59. Therefore, delocalized M(CO)₃-biguanide cations are an important pharmacophore for their selective accumulation in tumor cells with high mitochondrial membrane potential, enabling them to serve as both an imaging agent and a therapeutic agent.

### Example 12: Re-Phen and Phenformin Cell Assays

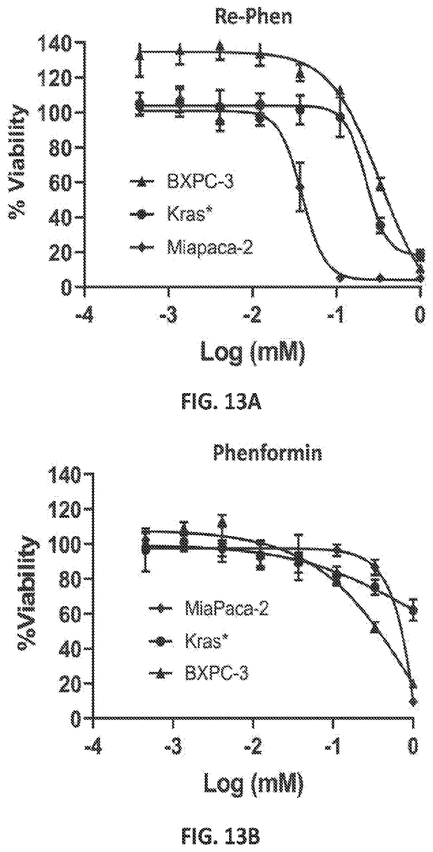

To determine cytotoxicity of Re-Phen and Phenformin, different cell lines were exposed to different concentrations of the compounds and their viabilities were followed over time. Pancreatic cells were seeded in 96-well plates (5000 cells/well) overnight, and treated with Re-Phen (FIG. 13A) or phenformin (FIG. 13B) in concentrations ranging from 4.57×10−4 mM to 1.0 mM at 37° C. for 72 h. Cell viability was measured using the MTT assay. The absorption at 590 nm was measured with a microplate reader (SpectraMax, Molecular Device, San Diego, CA) to quantify cell numbers. The proportion of cells was normalized to that of untreated cells and expressed as mean±standard error of the mean (SEM) (n=6). The half-maximal inhibitory concentration (IC₅₀) values were calculated by a sigmoidal dose-response (variable slope) curve-fitting using GraphPad Prism software. Results suggest that Re-Phen displays differential cytotoxicity against different PDAC cell lines. Further, results show that Re-Phen displays about 35-fold higher cytotoxicity against MiaPaca-2 PDAC cells (Table 1).

TABLE 1
Comparison of cytotoxicity of Re-Phen and phenformin
in a panel of pancreatic cancer cell lines.

| Cell Lines | BXPC-3 | Kras* | MiaPaca-2 |
|---|---|---|---|
| Re-Phen (IC₅₀ mM)) | 0.34 | 0.23 | 0.039 |
| Phenformin (IC₅₀ (mM)) | 1.1 | 0.75 | 13.6 |

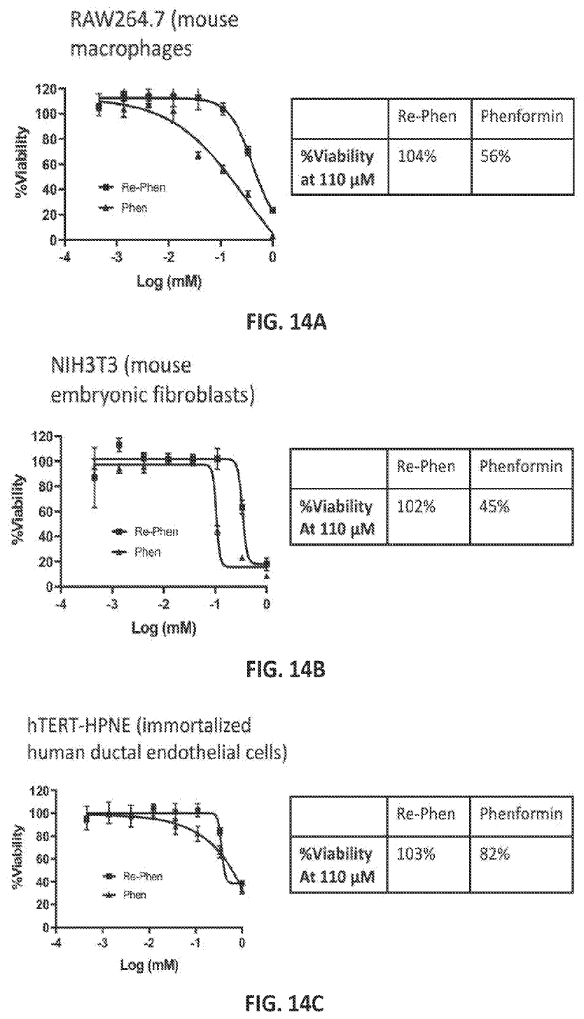

Next, RAW264.7 (FIG. 14A), NIH3T3 (FIG. 14B), or hTERT-HPNE (FIG. 14C) cells were seeded in 96-well plates (5000 cells/well) overnight, and treated with Re-Phen or phenformin in concentrations ranging from 4.57×10−4 mM to 1.0 mM at 37° C. for 72 hours. Cell viability was measured using the MTT assay. The proportion of cells was normalized to that of untreated cells and expressed as mean±standard error of the mean (SEM) (n=5). The half-maximal inhibitory concentration (IC₅₀) values were calculated by a sigmoidal dose-response (variable slope) curve-fitting using GraphPad Prism software. In sum, FIG. 14 shows that Re-Phen is generally less cytotoxic than phenformin against normal cells.

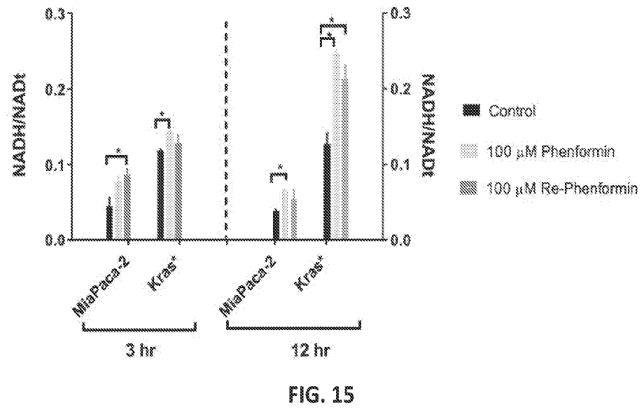

Following, PDAC cells were used to measure NADH-to-total NAD (NADt) ratios. NADH-to-total NAD (NADt) ratios were measured using a colorimetric kit (Biovision, Milpitas, CA). Briefly, cells were treated for different time periods as indicated and total NAD (NADt, NAD+NADH) was extracted following manufacturer provided protocol. For each of the extracted samples, half of the sample was heated at 60° C. for 30 minutes to decompose NAD+ while keeping NADH intact. Total NAD+ and NADH level was measured without the heating steps and in the presence of NAD cycling enzyme. The ratio of NADH to NADt were calculated on absorbance measured at 450 nm with a microplate reader (SpectraMax, Molecular Device, San Diego, CA). Cells treated with the same amount of DMSO used to prepare 100 μM phenformin or Re-Phen were used as controls. FIG. 15 shows a statistically significant difference in NADH/NADt ratio for DPAC cells treated with both phenphormin and Re-Phen as compared to control over three and twelve hours with a marked difference over longer exposure. Both phenformin and Re-Phen significantly increased NADH/NADt ratio (i.e., reduced NAD+/NADH ratio) in both PDAC cell lines at either three hour or twelve hour time points. NADH/NAD+ imbalance suggests that Re-Phen or phenoformin may play a role in reverse electron transfer (reduction of NAD+ to NADH by ubiquinol (CoQH₂)-derived electrons), which may be exploited to induce metabolic vulnerability.

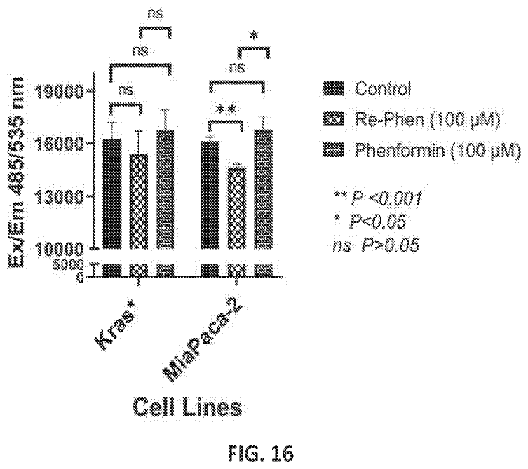

The production of intracellular reactive oxygen species (ROS) after treatments with Re-Phen and phenformin in PDAC cells was also measured (FIG. 16). DCFDA Cellular ROS Detection AssayKit (Abcam, Cambridge, MA) was used to measure Intracellular reactive oxygen species (ROS). Briefly, after seeded into 96-well plates at a cell density of 2.5×10⁴ cells per well overnight, cell monolayers were washed and incubated for 45 min at 37° C. in the dark in the presence of 25 μM DCFDA diluted in serum-free adhesion medium without phenol red according to the manufacturer's instructions. Cells were then treated with compounds as indicated for 1 hour. End-point fluorescence was measured in a fluorescence microplate reader (Syneregy H1, Bio-Tek, Winooski, VT) at 485 nm excitation and 535 nm emission wavelengths. Results show that Re-Phen but not phenformin reduced ROS levels in MiaPaca-2 cells, suggesting that Re-Phen inhibited reverse electron transfer (RET) in MiaPaca-2 cells.

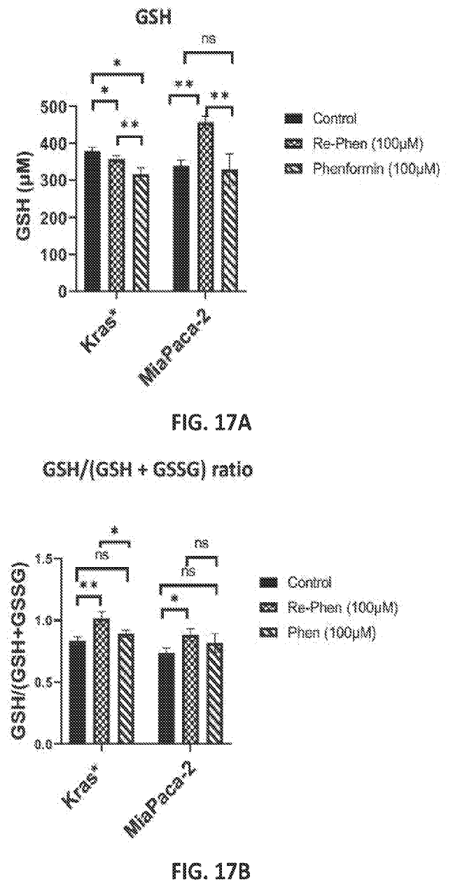

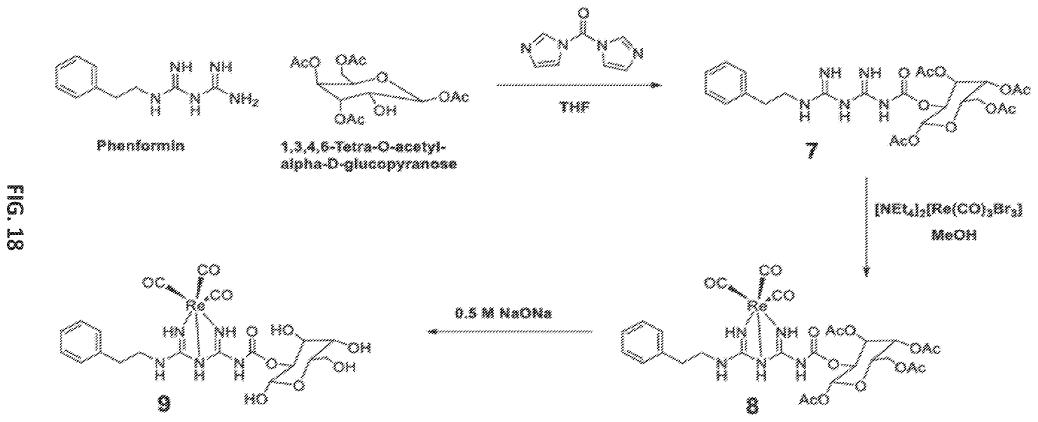

The production of intracellular glutathione (GSH) and GSH/GSSG ratio after treatment with Re-Phen and phenformin in PDAC cells was also compared (FIG. 17A and FIG. 17B). GSH/GSSG Ratio Detection Assay Kit (Abcam, Cambridge, MA) was used to measure Intracellular GSH/GSSG. Briefly, after seeded into 96-well plates at a cell density of 2.5×10⁴ cells per well overnight, the cells were pelleted by centrifugation. The pellet was lysed in mammalian lysis buffer and homogenized quickly by pipetting up and down a few times. After being centrifuged at top speed at 4° C. for 15 minutes, the supernatant was transferred to a fresh tube and used for deproteinization. The resultant supernatant was used for determining reduced GSH, total GSH and oxidized GSSG was calculated (Abcam, ab138881). Results suggest that in healthy cells, >90% of the total glutathione pool is in the reduced form (GSH) (GSH/GSSH≈10-100). In contrast, when cells are exposed to increased levels of oxidative stress, GSSG accumulates and the ratio of GSH to GSSG decrease. An increased ratio of GSH to (GSH+GSSG) is an indication of reduced oxidative stress.

The synthesis scheme of Re-Phen conjugates having a targeting moiety for targeted delivery is also shown (FIG. 18) and exemplified by the synthesis of a Re-Phen glucose carbamate conjugate. Briefly, the protected phenformin glucose conjugate 7 was synthesized by mixing equal molar ratio of phenformin, 1,3,4,6-Tetra-O-acetyl-alpha-D-glucopyranose and carbonyldiimidazole (CDI) in THF. The crude product was immediately acidified with 2N HCl and then conjugated with [NEt₄]₂[Re(CO)₃Br₃] in methanol. The Re conjugated urethane prodrug 8 underwent following basic deprotection step to remove the acetyl group to afford the Re-phenformin glucose conjugate 9. The reported method of FIG. 18 can be adapted for Re-Phen conjugates that form targeted delivery of Re-Phen to tumor cells and inflammatory cells involved in disease progression. For example, Re-Phen may be delivered to the brain for the treatment of neurodegenerative disease if it is conjugated to a compound that can cross the blood-brain barrier and accumulate in the neurons.

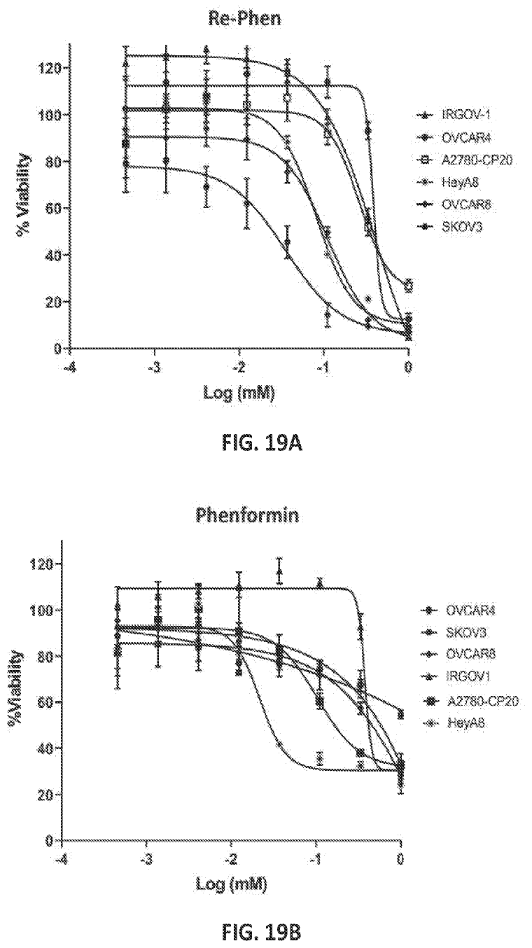

Finally, a comparison study of the cytotoxicity of Re-Phen and phenformin in a panel of ovarian cancer cell lines by MTT assay after a 72 hour incubation period was conducted. Ovarian cells were seeded at 5000 cells/well in 96-well plates overnight, and treated with Re-Phen or phenformin in different concentrations (from 4.57×10⁻⁴ mM to 1.0 mM in 3-fold increase) at 37° C. for 72 hours. Cell viability was measured using the MTT assay. The absorption at 590 nm was measured at microplate reader (SpectraMax, Molecular Device, San Diego, CA) to quantify cell numbers. The proportion of cells was normalized to that of untreated cells and expressed as mean±standard error of the mean (SEM) (n=5-6). The resulting reduced viability with half-maximal inhibitory concentration (IC₅₀) values of mM were calculated by a sigmoidal dose-response (variable slope) curve-fitting using GraphPad Prism software. The data are shown in FIG. 19A (Re-Phen) and FIG. 19B (phenformin). Results show that Re-Phen displays differential cytotoxicity against different ovarian cancer cell lines. In fact, Re-Phen is shown to display >2,000-fold and >5,000-fold higher cytotoxicity against OVACAR-8 and SKOV3 (Table 2) human ovarian cancer cell lines, respectively.

TABLE 2
Comparison of cytotoxicity of Re-Phen and phenformin
in a panel of ovarian cancer cell lines.

| Cell Lines | IRGOV-1 | OVCAR4 | A2789-cp20 | HeyA8 | OVCAR8 | SKOV3 |
|---|---|---|---|---|---|---|
| Re-Phen | 0.42 | 0.39 | 0.26 | 0.082 | 0.11 | 0.035 |
| (IC₅₀ mM)) | | | | | | |
| Phenformin | 0.38 | 2.43 | 0.10 | 0.022 | >200 | >200 |
| (IC₅₀ (mM)) | | | | | | |

All of the methods disclosed and claimed herein can be made and executed without undue experimentation in light of the present disclosure. While the compositions and methods of this invention have been described in terms of preferred examples, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A composition, comprising:
a compound of Formula I: 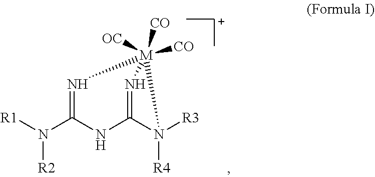
wherein:
M is a Group 7 transition metal,
R1 is selected from the group consisting of methyl, ethyl, propyl, butyl, and phenethyl;
R2 is selected from the group consisting of —H and methyl;
R3 is selected from the group consisting of —H, methyl, ethyl, propyl, butyl, and phenethyl; and
R4 is selected from the group consisting of —H, methyl, and -L-Z, wherein L is a linker and Z is a targeting unit.

2. The composition of claim 1, wherein M is selected from the group consisting of Tc and Re.

3. The composition of claim 1, wherein:
(a) R1 is methyl, R2 is methyl, R3 is —H, and R4 is —H;
(b) R1 is butyl, R2 is —H, R3 is —H, and R4 is —H;
(c) R1 is phenethyl, R2 is —H, R3 is —H, and R4 is —H; or
(d) R1 is phenethyl, R2 is —H, R3 is —H, and R4 is -L-Z, wherein L is carboxyl and Z is a 1,3,4,6-Tetra-O-acetyl-alpha-D-glucopyranose.

4. The composition of claim 1, wherein M is Re, R1 is phenethyl, and R2 is —H.

5. The composition of claim 1, further comprising a pharmaceutically-acceptable carrier.

6. The composition of claim 5, wherein the pharmaceutically-acceptable carrier is selected from the group consisting of polyethoxylated castor oil, ethanol, aqueous saline solutions, and mixtures thereof.

7. A method of treating cancer in a subject, comprising:
administering to a patient suffering from a cancer or suspected of suffering from a cancer a composition comprising a diagnostically-effective amount or a therapeutically-effective amount of a compound of Formula I: 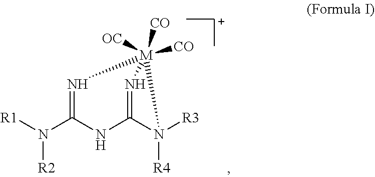
wherein
M is a Group 7 transition metal,
R1 is selected from the group consisting of methyl, ethyl, propyl, butyl, and phenethyl;
R2 is selected from the group consisting of —H and methyl;
R3 is selected from the group consisting of —H, methyl, ethyl, propyl, butyl, and phenethyl; and
R4 is selected from the group consisting of —H, methyl, and -L-Z, wherein L is a linker and Z is a targeting unit.

8. The method of claim 7, wherein M is selected from the group consisting of Tc and Re.

9. The method of claim 7, wherein:
(a) R1 is methyl, R2 is methyl, R3 is —H, and R4 is —H;
(b) R1 is butyl, R2 is —H, R3 is —H, and R4 is —H;
(c) R1 is phenethyl, R2 is —H, R3 is —H, and R4 is —H; or
(d) R1 is phenethyl, R2 is —H, R3 is —H, and R4 is -L-Z, wherein L is carboxyl and Z is a 1,3,4,6-Tetra-O-acetyl-alpha-D-glucopyranose.

10. The method of claim 7, wherein M is Re, R1 is phenethyl, and R2 is —H.

11. The method of claim 7, where in the composition further comprises a pharmaceutically-acceptable carrier.

12. The method of claim 11, wherein the pharmaceutically-acceptable carrier is selected from the group consisting of polyethoxylated castor oil, ethanol, aqueous saline solutions, any two thereof, and all three thereof.

13. The method of claim 7, wherein M is ^{99m}Tc, R1 is phenethyl, and R2 is —H, and the method further comprises:
imaging at least a portion of the patient's body after the administering.

14. The method of claim 13, wherein the imaging comprises computed tomography (CT) scanning.

15. The method of claim 7, further comprising:
administering, to the patient, a cancer treatment modality other than the compound of Formula I.

16. The method of claim 15, wherein the cancer treatment modality other than the compound of Formula I is selected from the group consisting of surgical resection, chemotherapy with a compound other than the compound of Formula I, immunotherapy, checkpoint inhibitor therapy, oncolytic virus therapy, thermal therapy, radiotherapy, and two or more thereof.

17. The method of claim 7, wherein the patient is a human being.

18. A kit, comprising:
a composition, comprising a diagnostically-effective amount or a therapeutically-effective amount of a compound of Formula I: 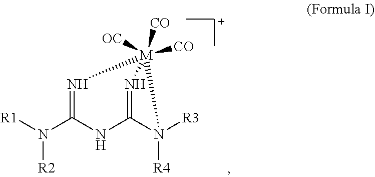
wherein
M is a Group 7 transition metal;
R1 is selected from the group consisting of methyl, ethyl, propyl, butyl, and phenethyl;
R2 is selected from the group consisting of —H and methyl;
R3 is selected from the group consisting of —H, methyl, ethyl, propyl, butyl, and phenethyl; and
R4 is selected from the group consisting of —H, methyl, and -L-Z, wherein L is a linker and Z is a targeting unit; and
instructions for use of the composition in a method comprising administering, to a patient suffering from a cancer or suspected of suffering from a cancer, the composition.

19. The kit of claim 18, wherein M is ^{99m}Tc, R1 is phenethyl, and R2 is —H, and the instructions further comprise instructions to image at least a portion of the patient's body after the administering.

20. The kit of claim 18, wherein M is Re, R1 is phenethyl, and R2 is —H.

21. (canceled)
